(19) United States
(12) Patent Application Publication
(10) Pub. No.: US 2016/0325058 A1
(43) Pub. Date: Nov. 10, 2016

(54) SYSTEMS AND METHODS FOR MANAGING PULMONARY MEDICATION DELIVERY

(71) Applicant: FocusStart Respiratory LLC, St. Paul, MN (US)

(72) Inventors: Serafin Samson, Maple Grove, MN (US); Daniel C. Sigg, St. Paul, MN (US); David Dieken, Minneapolis, MN (US); Seth Iverson, Glencoe, MN (US); Jacob Blank, St. Paul, MN (US); Paul Hattan, Minnetonka, MN (US)

(21) Appl. No.: 15/212,052

(22) Filed: Jul. 15, 2016

Related U.S. Application Data
(63) Continuation of application No. PCT/US2015/011864, filed on Jan. 16, 2015.
(60) Provisional application No. 61/928,306, filed on Jan. 16, 2014.
(60) Provisional application No. 62/023,694, filed on Jul. 11, 2014.

(51) Int. Cl. A61M 15/00; A61B 5/087; A61B 5/00; A61B 5/091

(52) U.S. Cl. CPC A61M 15/0065; A61M 15/0021; A61B 5/091; A61B 5/087; A61B 5/4839; A61B 5/742; A61B 5/4833; A61M 15/009; A61M 2205/3334; A61M 2205/52; A61M 2205/583; A61M 2205/18; A61M 2230/40; A61M 2205/581; A61M 2205/582; A61M 2205/3553; A61M 2205/502

(57) ABSTRACT

Example techniques and systems include managing delivery of a substance into an inhaled stream of air from a patient. For example, a system may include a housing configured to accept a medication canister containing a medication, the housing comprising a dispensing portion, a sensor configured to sense air flow within the dispensing portion, and a processor configured to transmit a signal indicative of the sensed air flow, wherein information associated with use of the pulmonary medication dosing device is generated by the computing device and based on the transmitted signal.

This application is a continuation of International Application No. PCT/US2015/011864, filed Jan. 16, 2015, which claims the benefit of U.S. Provisional Patent Application No. 61/928,306, filed Jan. 16, 2014, and U.S. Provisional Patent Application No. 62/023,694, filed Jul. 11, 2014. The entire contents of Application Nos. PCT/US2015/011864, 61/928,306, and 62/023,694 are incorporated herein by reference.

### TECHNICAL FIELD

The disclosure relates to medical devices and, more particularly, to the management of pulmonary disease by medical devices.

### BACKGROUND

Various diseases and disorders may be treatable with pulmonary medication or any other inhaled substances. For example, obstructive lung disease may cause narrowing of the airways that result in obstructions and a reduction in the volume of air obtained by the patient. In some cases, this narrowing may be the result of a variety of causes such as inflammation of airway walls, contraction of smooth muscle surrounding the airways, and/or excess mucus inside an airway. Relieving this obstruction may enable easier breathing for the patient and a greater volume of fresh air that reaches the alveoli (i.e., air sacs) of the lungs.

For conditions such as these, a patient may benefit from emergency and/or maintenance respiratory therapy through delivery of drug to the airway. This drug delivery and management of pulmonary condition, such as obstructive airway disease, may be achieved through the use of an inhaler device. An inhaler device may be referred to as a metered dose inhaler in some examples. A patient may operate the inhaler device by placing the mouthpiece of the device inside the patient's mouth and inhaling while depressing a canister containing pressurized medication. Depression of the canister opens a metered valve of the canister such that the medication is released into the air flow of the inhalation of the patient.

### SUMMARY

Generally, this disclosure describes various techniques and systems for managing delivery of a substance into an inhaled stream of air from a patient as well as to manage disease progression, disease treatment, and therapy compliance. For example, a pulmonary medication dosing device (PMDD) may be configured to sense air flow indicative of inhalation (e.g., the start or initiation of an inhalation of air by the patient) and/or detect when medication is delivered from a medication canister associated with the PMDD. This information may, for example, be transmitted to another computing device (e.g., a mobile computing device) that generates and/or delivers an alert that instructs a patient when to actuate the medication canister and/or generate a log of medication delivery from the PMDD (e.g., an e-diary). A computing device may use the flow information to measure pulmonary function of the patient at a given point and time; the computing device may transmit this information to a health care provider

The PMDD may transmit information related to when an actuation occurred. This actuation information may be received and analyzed by a computing device and compared to air flow information also received and indicative of the air flow within the PMDD associated with the actuation. The computing device may correlate the timing of the sensed air flow (e.g., air flow rate) with the timing of the actuation and determine, based on one or more algorithms, whether the actuation was properly timed to the air flow of the inhalation or improperly timed to the airflow of the inhalation. The computing device may also generate an indication of this proper, or improper, timing for presentation to the patient. For example, the computing device may indicate whether an actuation was proper or improper and/or present a graphical representation of the sensed air flow and actuation timing. In some examples, the PMDD may, alternatively or additionally, present an indication of the determination of whether the actuation was proper or improper. The patient may use this information to improve actuation timing. In some examples, the computing device may receive information indicative of environmental conditions and/or patient conditions that may affect the pulmonary function of the patient. The computing device may store this information and/or correlate the information with actuation information such that the patient or clinician can determine if medication was needed due to an environmental or patient condition or otherwise manage the patient disease.

In one example, the disclosure describes a method that includes sensing, by a sensor, air flow within a portion of a pulmonary medication dosing device and transmitting, by a processor, a signal indicative of the sensed air flow, wherein an alert that instructs a user to actuate a medication canister associated with the pulmonary medication dosing device is generated based on the transmitted signal.

In another example, the disclosure describes a system that includes a housing configured to accept a medication canister containing a medication, the housing comprising a dispensing portion, a sensor configured to sense air flow within the dispensing portion, and a processor configured to transmit a signal indicative of the sensed air flow, wherein an alert that instructs a user to actuate a medication canister associated with the housing is generated based on the transmitted signal.

In another example, the disclosure describes a method that includes receiving, by a processor of a computing device, a signal indicative of sensed air flow within a portion of a pulmonary medication dosing device in communication with the processor and generating, by the processor and based on the signal, an alert that instructs a user to actuate a medication canister associated with the pulmonary medical dosing device.

In another example, the disclosure describes a computing device that includes one or more processors configured to receive, from a pulmonary medication dosing device, a signal indicative of sensed air flow within a portion of the pulmonary medication dosing device and generate, based on the signal, an alert that instructs a user to actuate a medication canister associated with the pulmonary medication dosing device.

In another example, the disclosure describes a device configured to sense air flow within a portion of a pulmonary medical dosing device, transmit information indicative of the sensed air flow to a processor, and, based on the information, determine one or more pulmonary functions. The sensed air flow information may be displayed to the user, stored, and also transmitted via the computing device (e.g. a mobile handheld device) to a cloud-based storage system via a network. The sensed air flow information may also be transmitted to a health care provider associated with the patient. The patient or health care provider may use the sensed air flow information to manage both compliance of the user to delivery of the prescribed medication, and, in some examples, the efficacy of the prescribed pulmonary medication in a cost-efficient manner.

### BRIEF DESCRIPTION OF DRAWINGS

FIGS. 1A-1D are a conceptual drawings illustrating an example system that includes a respective pulmonary medication delivery device (PMDD) and a computing device in relation to a user.

FIG. 2 is a conceptual drawing illustrating an example PMDD that includes a flow sensor.

FIG. 3A is a conceptual drawing illustrating an example PMDD that includes a canister actuation sensor.

FIG. 3B is a conceptual drawing illustrating an example PMDD that includes a flow sensor positioned within the PMDD housing.

FIG. 4 is a functional block diagram illustrating an example configuration of the PMDD of FIG. 1A.

FIG. 5 is a functional block diagram illustrating an example configuration of the computing device of FIG. 1A.

FIG. 6 is a flow diagram of an example process for detecting air flow and canister actuation associated with a PMDD.

FIG. 7 is a flow diagram of an example process for generating an alert that instructs a user to actuate a PMDD.

FIG. 8 is a conceptual diagram of an example system that includes a computing device and a networked server associated with use of a PMDD.

FIG. 9 is a flow diagram of an example process for monitoring the number of medication doses received by a patient.

FIG. 10 is a flow diagram of an example process for monitoring the timing of actuations used to deliver medication doses to a patient.

FIG. 11 is a flow diagram of an example process for monitoring lung function of a patient.

FIG. 12 is a flow diagram of an example process for monitoring one or more environmental conditions relevant to a patient.

FIG. 13 is a flow diagram of an example process for determining compliance thresholds for a patient.

FIG. 14 is a conceptual diagram of an example user interface providing flow and actuation data related to patient initiated actuations.

FIG. 15 is a conceptual diagram of an example user interface providing qualified actuation information of actuations over time.

FIG. 16 is a conceptual diagram of an example user interface providing an indication of effective actuation timing for a detected inhalation of a patient.

### DETAILED DESCRIPTION

This disclosure describes various techniques and systems for managing the delivery of a substance (e.g., a medication) to a patient and/or managing a patient disease (e.g., a pulmonary function disease or disorder such as asthma or chronic obstructive pulmonary disease). Typically, patients requiring pulmonary medications to treat various disorders and conditions may rely upon metered dose inhalers (MDIs) or other delivery devices to obtain the prescribed pulmonary medication. To receive medication from an example MDI, a patient attempts to coordinate an inhaled breath with the manual depression of a medication canister coupled to a housing of the MDI. Efficacy of medication delivery using an MDI may depend on the ability of the patient (or caregiver) to accurately coordinate breathing and hand manipulation functions to actuate the medication canister associated with the inhaler.

Generally, this disclosure describes various techniques and systems for facilitating and/or monitoring delivery of medication from an MDI, e.g., a pulmonary medication dosing device (PMDD). For example, a system may provide breath information to a user of a PMDD in response to detecting air flow. In one example, a PMDD may be configured to sense air flow indicative of inhalation (e.g., the start or initiation of an inhalation of air by the patient) and generate a signal indicative of the sensed air flow. This signal may be used to provide an alert (e.g., a type of breath information) to the patient, or another user such as a caregiver, that instructs the patient when to depress, or actuate, a medication canister associated with the PMDD during the patient inhalation. The PMDD may transmit this signal indicative of the air flow to another computing device (e.g., a mobile computing device such as a smartphone, tablet computer, notebook computer, or portable medical device). Therefore, in response to generating the signal indicative of the air flow, the PMDD may transmit the signal to the computing device, and the computing device may analyze the signal and generate the alert that instructs actuation of the medication canister of the PMDD and deliver the alert to the patient or user via one or more output devices. The alert may be in the form of one or more of a visual cue, an audible cue, or a tactile cue. The computing device may present the alert to the user. In other examples, the PMDD may be configured to generate and/or deliver an alert. Such a tool can also be used as a training tool to train users in the correct usage of PMDDs, in the presence or absence of a health care provider. In this manner, the alerts and/or training tools described herein may allow the patient and/or health care professional to improve the efficacy of drug delivery and ensure that the patient is receiving the prescribed dosage of medication. In other words, even if the patient is attempting to take medication at the prescribed times, ineffective or improper delivery of medication may result in non-compliance with the prescribed dosage of medication.

In addition, or alternatively, the PMDD may detect when a substance is delivered to the patient from the medication canister associated with the PMDD. For example, a sensor (e.g., a flow sensor and/or pressure sensor) may be disposed within the path of the delivered substance (e.g., within a housing or mouthpiece of the PMDD) to detect the delivery of the medication to the patient. In this manner, the sensor may be disposed within the vicinity (e.g., within an area in which medication can be detected) of the delivery of the medication, directly within the medication path, or otherwise disposed in a position selected to enable the sensor to detect when a substance is delivered. In another example, a sensor (e.g., a pressure switch) may be disposed in or on the PMDD and adjacent to the medication canister such that the sensor is configured to detect when the medication canister is depressed or actuated. The PMDD may transmit a signal indicative of the canister actuation (e.g., medication delivery) to another computing device (e.g., a mobile computing device and/or other devices). This signal may be used to generate a log of dispensed medication from the medication canister or otherwise track the delivery of medication to the patient from the PMDD. In some examples, the computing device may receive a user input that indicates medication has been delivered and/or the canister has been actuated.

In another example, the system may present the breath information as a type other than an alert. For example, the breath information may be presented in the form of data, graphs, etc. related to an air flow parameter representative of an inhalation and/or an inhalation associated with the PMDD. In some examples, the system may display a graph illustrating the actuations of medication from the PMDD and whether or not the actuation was determined to be effective (i.e., “good”) or ineffective (i.e., “bad”) and transferring the medication of the dose to the patient. The device may be configured to sense the respiratory function of the user via sensing air flow. For example, the sensor of a PMDD and a separate computing device (e.g. a mobile computing device) can be configured to measure peak expiratory flow. Flow information, but also other important information (e.g., time, date, location, last medication application, etc.) can be stored on the computer device (e.g. mobile computing device), and from time to time transmitted via a network to a networked server or cloud-based storage system. The computing device or networked device can be configured to transmit the information, periodically, continuously, or from time to time, to a health care provider of the patient. In addition, the computing device may present (e.g., display) one or more air flow parameters to the user of the PMDD in response to the detection of air flow. This feedback may be provided to allow the patient to manage when to actuate a medication canister and/or manage breathing function of the user of the PMDD (e.g., the patient).

In some examples, the PMDD sensor may include a spindle or other rotating device, a vane, or pressure sensor configured to convert a mechanical deformation due to the air flow to an electrical signal indicative of the air flow. In this manner, a magnitude and/or direction of air flow within the PMDD or indicative of patient breath may be detected by sensing flow of fluid or gas and/or changes in pressure at one or more locations. One or more of these sensors may be configured to detect air flow and/or medication delivery from the medication canister associated with the PMDD.

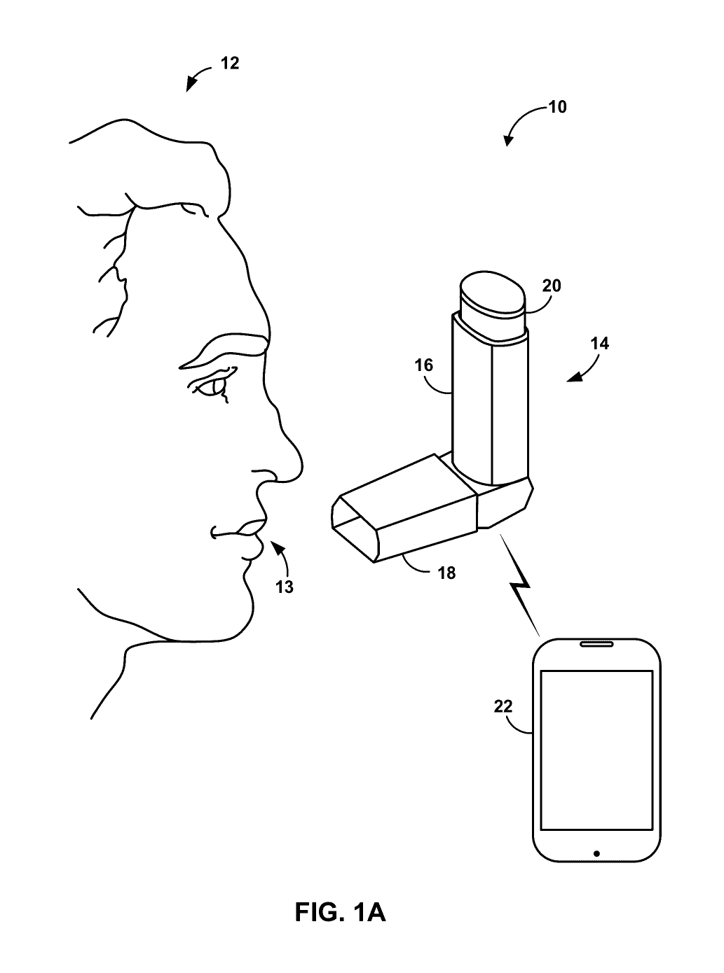

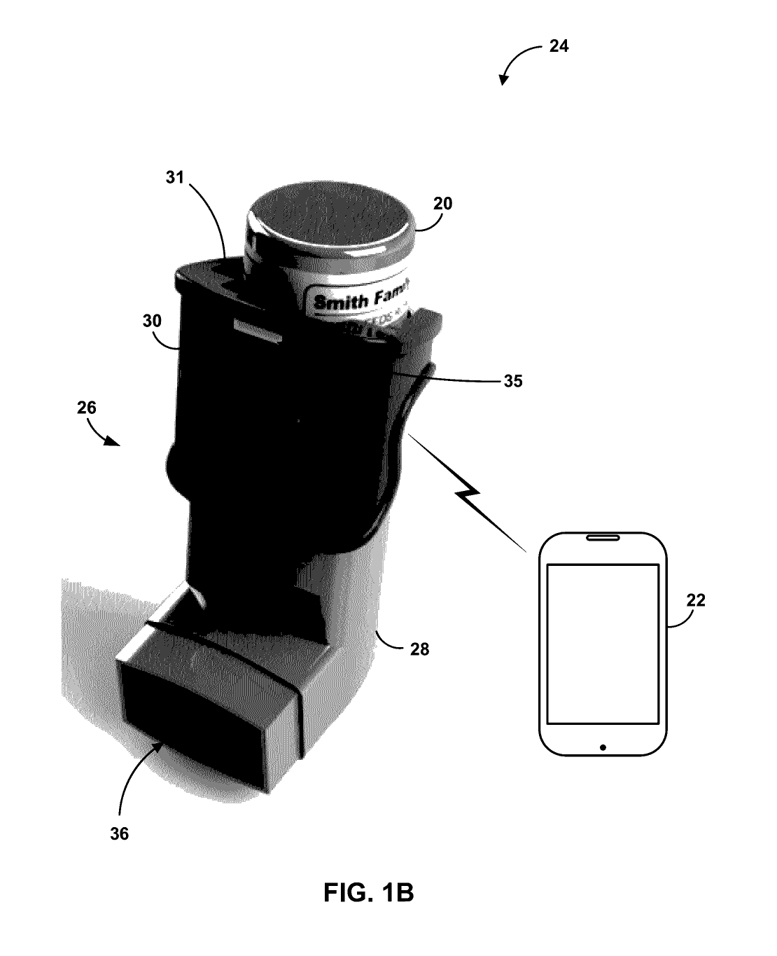

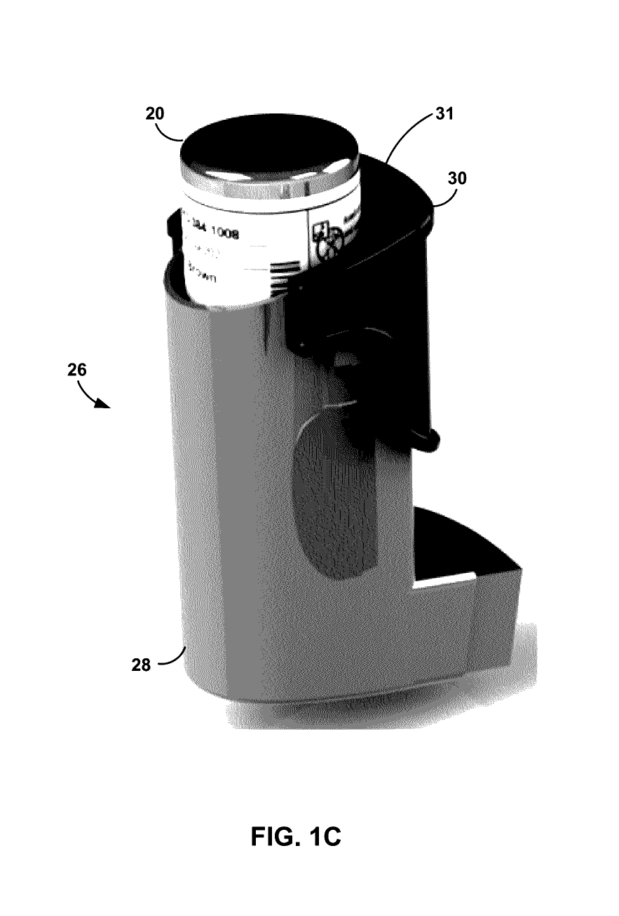

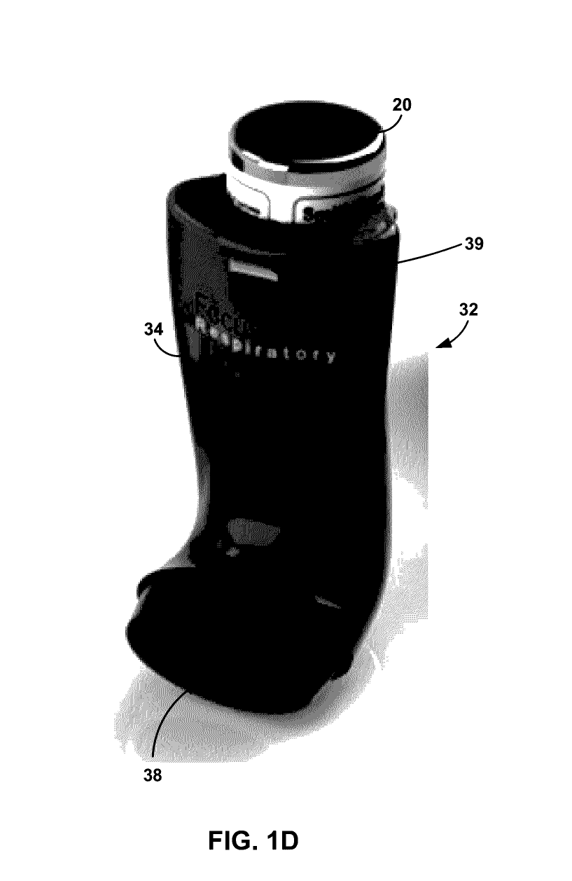

Although the PMDD may be described as generally including components to provide the functionality described herein, the PMDD may include a housing typical for MDIs and an add-on device that includes one or more sensors and other electronics necessary to perform the functions described herein. The add-on may take the form of a mouthpiece configured to be coupled to an MDI housing that accepts a medication canister. In this manner, the add-on portion of the PMDD may be an additional device that the patient may add to a previously used MDI. In some cases, sensors and/or electrodes pre-fabricated within the housing and/or add-on component. As described herein, the add-on component may be different than the mouthpiece. For example, the add-on may be an assembly configured to be attached to a portion of the PMDD separate from the medication dispensing region of the PMDD. In one example, the housing may incorporate the mouthpiece portion (i.e., an outlet of the housing) with the add-on assembly attachable to an intake portion of the housing (e.g., PMDD 26 of FIG. 1B).

Although this disclosure is generally described with respect to pulmonary medications, the techniques and devices described herein may be used for delivery of any substance in response to detecting an air flow. For example, a patient may use a PMDD to receive medication directed to the mouth, throat, nasal passages, etc.

The sensor may be configured to use detected airflow to generate measured air volumes (or provide data usable for generating air volumes from the air flow data. For example, the sensor may detect, measure, and transmit to another computing device (e.g., from PMDD to a mobile computing device) inspiratory or expiratory volumes. The computing device may measure, display, and transmit Forced Vital Capacity (FVC) (i.e. the largest amount of air that user can exhale from the lungs after inhaling the largest breath possible), or Forced Expiratory Volume (FEV1) (i.e. the amount of air the user can exhale from the lungs within the first second of exhalation).

FIG. 1A is a conceptual drawing illustrating an example system 10 that includes a pulmonary medication delivery device (PMDD) 14 and computing device 22 in relation to user 12. PMDD 14 may include a mouthpiece 18 and housing 16. Mouthpiece 18 may be removably attached to housing 16, permanently coupled to housing 16, or formed with housing 16, for example. Housing 16 may be configured to accept canister 20, wherein canister 20 includes a medication to be dispensed to patient 12 via PMDD 14. Canister 20 may be replaceable or exchangeable such that PMDD 14 can accept a variety of different canisters containing any number of different medications. In some examples, housing 16 may be specific to a particular canister shape and size and/or medication. For example, an orifice within housing 16 that dispenses the medication may be shaped and sized to produce a medication cloud appropriate for the particular medication and/or prescribed dosing. A large patient population may benefit from respiratory therapy, either maintenance therapy or emergency therapy, through the delivery of medication to airways through the use of inhaler devices.

PMDD 14 and system 10 may be used to achieve improved delivery of pulmonary medication (or any other airborne substance) to patient 12. The respiratory tract can be considered as a filter that removes particles from inspired air. The effectiveness of this filter depends on particle properties (e.g. size, shape, density, and charge), respiratory tract morphology, and the breathing pattern (e.g. airflow rate and tidal volume) of patient 12. These various factors may determine the quantity of particles that are deposited in the respiratory tract and in what region of the respiratory tract the particles are deposited. For example, the density of an inhaled gas may influence deposition of aerosol substances in the lung. Moreover, high inspiratory airflows often employed by patient 12 during use of inhaling devices (e.g., MDIs or nebulizers). These high air flows are typically associated with higher turbulence, but inhalation of a less dense gas can contribute to less turbulent, and more laminar, airflow within the respiratory tract.

To achieve an adequate therapeutic effect from inhaled medication, a sufficient deposition of medication must be made in the medium and small airways of patient 12. This desired result may require a competent inhalation technique if the delivery device is patient-activated, irrespective of the design and relative complexity of any specific manual inhalation device. Prompted coordination of inhalation of patient 12 and actuation (e.g., depression) of canister 20 may increase the amount of medication delivered from canister 20 to the lungs of patient 12 may increase the therapeutic effect derived from the use of PMDD 14.

Patient 12 may use PMDD 14 to deliver a dose of medication from canister 20 into the lungs of patient 12. Patient 12 may place a portion of mouthpiece 18 into mouth 13. In some examples, mouthpiece 18 may be referred to a dispensing portion of PMDD 14, and mouthpiece 18 may include or define a nozzle within which air flows into patient 12. Mouthpiece 18 may be attached to housing 16 via a latch or press-fit, for example, or mouthpiece 18 may be formed as part of housing 16. With canister 20 depressed such that a metered valve of canister 20 is open, patient 12 may begin to inhale air through an interior channel formed by mouthpiece 18. In response to this inhalation, a sensor (not shown) within PMDD 14 may generate a signal indicative of the air flow of the inhalation toward patient 12. Based on the generated signal, a processor of PMDD 14 and/or computing device 22 may generate and/or deliver an alert that instructs patient 12 when to depress or actuate canister 20 during the inhalation of patient 12. In this manner, computing device 22 and/or PMDD 14 may instruct or prompt patient 12 to actuate canister 20 based on the sensed air flow of the inhalation.

Canister 20 may include a metering valve that controls the release of medication from canister 20. The metering valve may include a spring or other mechanism that configures the metering valve in a closed state without any external forces acting on the metering valve. In response to depressing the metering valve (e.g., compressing the spring of the metering valve) the metering valve may open to release a predetermined dose of the medication. When coupled to PMDD 14, patient 12 (or another user such as a caregiver) may use one or more fingers, or a hand, for example, to manipulate canister 20 and depress canister 20 with respect to housing 16. Canister 20 may include different mechanisms for dispensing different types of medications. For example, a metering valve may be appropriate for a liquid medication, but other mechanisms may be appropriate for dry powder medications. PMDD 14, or any other PMDD described herein, may be configured to function with any type of medication and dispensing mechanism.

In one example, PMDD 14 or system 10 may generate a signal indicative of air flow within a portion of PMDD 14. PMDD 14 may include a sensor configured to generate the signal. The air flow may be present within mouthpiece 18 and/or housing 16. In some examples, the generated signal indicative of air flow may already be calibrated or in a form that can be interpretable to represent some parameter of the air flow. For example, the signal may be an analog electrical signal or raw data. In other examples, a sensor module or processor may determine, based on the signal, a parameter value representative of the signal and including a usable value representing one or more conditions of the air flow. In some examples, the parameter value may be indicative of one or more of a flow rate (e.g., cubic meters per second), a velocity (e.g., meters per second), or a pressure of the air through a portion of PMDD 14.

PMDD 14 and/or computing device 22 may generate the alert that instructs patient 12 to actuate canister 20, or computing device 22 may record, and transmit parameters of pulmonary function as discussed above. In one example, PMDD 14 may transmit the signal indicative of air flow to computing device 22, and computing device 22 may generate the alert. Computing device 22 may then deliver the alert via one or more output devices configured to deliver a visual cue, an audible cue, and/or a tactile cue. In another example, PMDD 14 may generate the alert and transmit the alert to computing device 22, and computing device 22 may delver the alert via one or more output devices. In another example, computing device 22 may transmit the alert to PMDD 14, and PMDD 14 may be configured to deliver the alert via one or more of a visual cue, an audible cue, and/or a tactile cue. In this manner, computing device 22 and/or PMDD 14 may deliver at least a portion or the entire alert to patient 12.

For example, computing device 22 may generate, based on a value of the signal indicative of the air flow, when or whether to generate the alert. In one example, computing device 22 may generate the alert in response to determining that a value of the signal has exceeded a threshold. A threshold may be used by a processor to determine when an inhalation has occurred instead of a non-inhalation movement of air. For example, the threshold may be a threshold flow rate, velocity, or pressure. In response to determining that the detected parameter value is above the threshold, the computing device 22 may generate an alert for delivery to patient 12 that instructs patient 12 to actuate canister 20 and deliver medication to patient 12 during the inhalation.

In some examples, the sensor of PMDD 14, e.g., an air flow sensor or pressure sensor, may generate an electrical signal indicative of the air flow within the portion of PMDD 14 at which the sensor is located (e.g., mouthpiece 18 or housing 16). This electrical signal may be generated as a function of a deformation of an element of the sensor or a mechanical motion of the element of the sensor. Alternatively, the electrical signal may be generated as a function of any change in current in an electrical element (e.g., a thermocouple or a thermistor) of the sensor due to air flow that cools the element. Any of the air flow sensors or pressure sensors described herein may also be configured to detect the presence of a delivered medication from canister 20.

As shown in the example of FIG. 1A, PMDD 14 is in communication with computing device 22. Computing device 22 may be configured to receive data from PMDD 14, generate alerts (e.g., prompts) that instruct patient 12 to actuate canister 20 and/or deliver the alerts to the patient. PMDD 14 may include a communication unit configured for, in response to generating the signal indicative of the air flow within PMDD 14, transmitting the signal to a communication unit of computing device 22. In response to receiving the generated signal from the PMDD 14, computing device 22 may generate the alert for delivery to patient 12 (or another user) that instructs patient 12 to depress (e.g., actuate) canister 20, and, in some examples, deliver the alert to patient 12 via one or more output devices (e.g., one or more displays, lights, speakers, haptic feedback devices, etc.). In some examples, computing device 22 may first determine a parameter value representative of the signal and/or compare the signal or parameter value to a respective parameter. In this manner, computing device 22 may generate the command based on a parameter value representing the air flow, such as velocity, flow rate, pressure, etc.

PMDD 14 may also detect when canister 20 is actuated and/or medication is delivered to the patient. PMDD 14 may include one or more sensors that detect the actuation of canister 20 and/or the delivery of medication out of canister 20. In some examples, the same sensor that senses air flow from inhalation may detect the delivery of medication from canister 20. PMDD 14 may transmit a signal indicative of medication delivery and/or canister actuation to computing device 22. In response to receiving the signal, computing device 22 may store the signal as an entry in a log of medication delivery events. Computing device 22 may store the delivery log and/or transmit data from the log to other devices for review and monitoring of medication delivery from PMDD 14. In some examples, computing device 22 may generate and deliver messages based on the timing, frequency, or number of times medication has been delivered from canister 20 and PMDD 14. For example, a message may remind patient 12 to administer another dose of medication from canister 20 or, responsive to detecting an inhalation, warn patient 12 from administering any more medication in an attempt to minimize possible overdosing of medication.

Computing device 22 may include one or more applications that control one or more modules configured to perform various functions related to the control of the PMDD 14, such as a sensor module that determines parameter values and an alert module that generates alerts that instruct patient 12 to actuate canister 20 or otherwise administer medication. These one or more applications may be included in a computer-readable storage medium (e.g., storage device of computing device 22). In this manner, the applications may include instructions that, when executed by one or more processors of computing device 22, cause the one or more processors to perform the various functions described herein.

Although PMDD 14 may generally include electronic components that facilitate communication with computing device 22 to generate and/or deliver the alerts, PMDD 14 may independently include processors and/or modules configured to provide the functionality of computing device 22. For example, PMDD 14 may receive the signal indicative of air flow, generate the alert, and deliver the alert to patient 12 that instructs patient 12 to actuate canister 20.

PMDD 14 may include one or more air flow sensors, or electrical components within housing 16 and/or mouthpiece 18. For example, mouthpiece 18 may include the sensor and associated electronics for controlling the sensor and, in some examples, communicating with computing device 22. In this manner, housing 16 may be modified from currently available MDIs available for manual dispensing of medication.

Alternatively, mouthpiece 18 may include the functionality described herein as an add-on device to unmodified MDIs (e.g., a commercially available MDI housing). In this manner, housing 16 may be an unmodified housing of typical MDIs for manual dispensing of medication, and mouthpiece 18 may be added as needed to achieve the features described herein. Mouthpiece 18 may be constructed to be removably coupled to a mouthpiece portion, or exit portion, of housing 16. In addition, mouthpiece 18 may include the air flow sensor and any necessary electronics such as a power source and communication unit. Medication from canister 20 may typically be dispensed into an air flow through the orifice.

PMDD 14 and computing device 22 may not need to be coupled together for transmitting data between devices. For example, data may be transmitted across a sufficient distance such that computing device 22 may remain in an article of clothing of patient 12 or otherwise in the vicinity of PMDD 14. In other examples, a flexible connecting band may be used to couple PMDD 14 to computing device 22. In some examples, the flexible connecting band may include one or more wired connections for communication between PMDD 14 and computing device 22. In other examples, a separate wired connection may be provided, or PMDD 14 and computing may communicate via one or more wireless communication protocols. Wireless communication may utilize Bluetooth, near-field communication, infrared communication, WiFi protocols, or any other wireless communication standard.

Computing device 22 may utilize any number of additional functions or capabilities to facilitate delivery of medication to patient 12, store usage history of PMDD 14, inform clinicians of changes to medication use, notify patient 12 when to take medication based on a schedule, or notify patient 12 of changes to usage instructed by a clinician. Computing device 22 may also utilize a global position system (GPS) system to identify the location of patient 12 and, based on known high pollution and/or low air quality areas and/or potentially dangerous high altitudes with lower partial pressures of oxygen, notify patient 12 to preemptively take another dose of medication. Computing device 22 may also notify a health care provider (e.g., a clinician) of the patient's usage history. Computing device 22 may transmit data to the health care provider via a network (e.g., wireless networks, cellular networks, the Internet, or any other means) to facilitate remote monitoring of patient condition or compliance by the health care provider. In response to such monitoring, the health care provider may transmit information to computing device 22 and/or the patient to optimize medication administration or other therapies according to patient usage patterns and clinical symptoms.

In other examples, PMDD 14 may be constructed for different breathing situations. For example, PMDD 14 may include an apparatus to direct the air flow to the nose or nose and mouth (e.g., an inhalation mask). PMDD 14 may alternatively be constructed to direct the medication into a breathing circuit or breathing tube that may be used when patient 12 is intubated. In this manner, PMDD 14 and, in some examples, computing device 22 may be configured to deliver medication to any type of air flow during a patient inhalation.

Canister 20 may contain medication in any number of forms. Canister 20 may contain a pressurized substance, non-pressurized liquid, or a powder substance, for example. When canister 20 contains a pressurized substance, the pressurized substance may be released through an orifice pass area that creates an aerosol. In the case of a non-pressurized substance, PMDD 14 may include an electro-mechanical or active mechanism configured to inject or otherwise provide the substance into the stream of air. A perforated barrier may be selectively placed in housing 16 to contain the particles of medication. For example, the mechanism or force, along with the barrier, creates a process that atomizes the aerosol particles of the substance (e.g., into a soft mist) to facilitate a more efficient absorption of the substance in the lower airways. The configuration of the barrier may at least partially control the particle size intended for patient 12.

In this manner, PMDD 14 may be configured to deliver various substances to patient 12. For example, PMDD 14 may be configured to aerosolize a substance originating from a pressurized canister. PMDD 14 may aerosolize the substance from a pressurized or non-pressurized canister in the form of soft mist. PMDD 14 may also be configured to aerosolize a substance originating from a non-pressurized liquid canister. PMDD 14 may alternatively be configured to dispense a substance in the form of dry powder, originating from a dry powder inhaler or canister. According to these descriptions, in some examples, mouthpiece 18 may be configured to couple with a pressurized MDI, a dry powder inhaler (DPI), a small-volume nebulizer (SVN), or a soft mist inhaler (SMI). In this manner, the systems, devices, and techniques described herein related to medication delivery applies to liquid, powdered, and vaporized medication delivery or any other type of inhaled medication.

The substance delivered to patient 12 using PMDD 14 may include any medication, drug, potentially therapeutic substance, or any combination thereof. Example substances may include beta-adrenergic agonists (e.g., terbutaline), chromoglycin, anticholinergic drugs, and various steroids. In some examples, the substance delivered to patient 12 using PMDD 14 may include any vaccine with or without carriers and/or adjuvant, or any combination thereof, insulin, or any other compound deliverable to the nasal passages, mouth, or lungs. In other examples, the substance selected for delivery to patient 12 using PMDD 14 may be used for the treatment of any condition. Example conditions may include asthma and chronic obstructive pulmonary disease.

In some examples, the substance selected for delivery to patient 12 using PMDD 14 may delivered into the airway of patient 12 in a form selected from the group including a powder, a granule, a cachet, a capsule, a tablet, a paste, a cream, a gel, an ointment, a salve, a foam, a paste, a lotion, a cream, an oil suspension, a spray, a suspension, a solution, an emulsion, a patch, a stick, a spray, preferably a nasal spray, or a buccal spray, a mouth wash, an aerosol, in a venture effect, a drink, and a solution. PMDD 14 may include an aperture or active device to facilitate the delivery of such a form of the substance.

Housing 16 and/or mouthpiece 18 may be constructed of a polymer, composite, metal alloy, or any combination thereof. The materials used to construct housing 16 and/or mouthpiece may be selected not to react with any medication to be dispensed from canister 20. In this manner, the materials may be selected to be bio-compatible, and in some examples, sterilizable. Although housing 16, mouthpiece 18, and any other components contained therein (e.g., one or more sensors and electronics) may be constructed to be reusable between multiple different canisters 20, for example, any or all of PMDD 14 may be constructed to be disposable.

PMDD 14 may be constructed with one or more components of which one or more of the components may be disposable (e.g., constructed to be disposed of when medication runs out or after any other defined use period for the patient) and/or one or more of the components may be intended to be non-disposable (e.g., constructed to be transferred for use between different medication canisters and/or different users). In one example, a reusable assembly (e.g., attachable assembly 30 of FIG. 1B) that includes a flow sensor may clip onto or otherwise attach to a disposable housing 16, where disposable housing 16 may be a commonly-used disposable metered-dose inhaler housing. The reusable assembly may include the flow sensor and/or necessary electronics for transmitting flow data from the flow sensor to another device, such as computing device 22. In this manner, the reusable assembly may be transferable between different disposable housings 16.

In another example, PMDD 14 may include a non-disposable housing 16 that attaches to a disposable mouthpiece portion (e.g., similar to mouthpiece 18). A disposable mouthpiece may prevent excess buildup of medication over time. In this example, the mouthpiece portion may include a flow sensor that detects air flow to or from the patient 12. The mouthpiece portion may include an electronics package that includes a communication unit for transferring data from the flow sensor and, in some examples, a power source. Alternatively, the non-disposable housing 16 may include the electronics package and power source, and housing 16 and the disposable mouthpiece may include electrical connections that mate when the mouthpiece is attached to housing 16 such that the flow sensor in the mouthpiece communicates with the electronics package in housing 16. Alternatively, the flow sensor may be located within the non-disposable housing but mechanically separated from the medication and/or condensation by being placed within a tube or other constriction for the air flow. In this manner, the flow sensor may detect inhalation and/or exhalation air flow from the patient and be protected from possible damage due to medication coating the flow sensor.

In some examples where the flow sensor is replaceable or permanently attached to a disposable portion of PMDD 14, computing device 22 or electronics within PMDD 14 may determine when the flow sensor needs to be replaced. For example, performance of the flow sensor may be reduced after medication is disposed on the sensor element or mechanism, an impact to PMDD 14 damages the flow sensor, moisture from patient breath, or any other cause. In one example, computing device 22 may compare acceleration data from an accelerometer disposed within PMDD 14 to air flow data from the air flow sensor (e.g., air should move across the flow sensor during PMDD 14 movement) during shaking of PMDD 14 or other movement. Computing device 22 may determine that the flow sensor needs to be replaced when air flow direction and/or magnitude no longer corresponds to the data received from the accelerometer. In other examples, computing device 22 may compare flow data from redundant flow sensors and determine that at least one flow sensor needs replacing when the air flow varies between the sensors greater than a threshold percentage or threshold magnitude. In some examples, computing device 22 may track the number of actuations of PMDD 14, inhalations and/or exhalations, and/or time from first use of the flow sensor, compare the tracked data to a respective threshold, and determine that the flow sensor requires replacing after the flow sensor use as exceeded the threshold. In any event computing device 22 may, responsive to determining that a flow sensor needs to be replaced, generate, for display to the user, an alert to the user that the flow sensor (or portion of PMDD 14 containing the flow sensor) needs to be replaced.

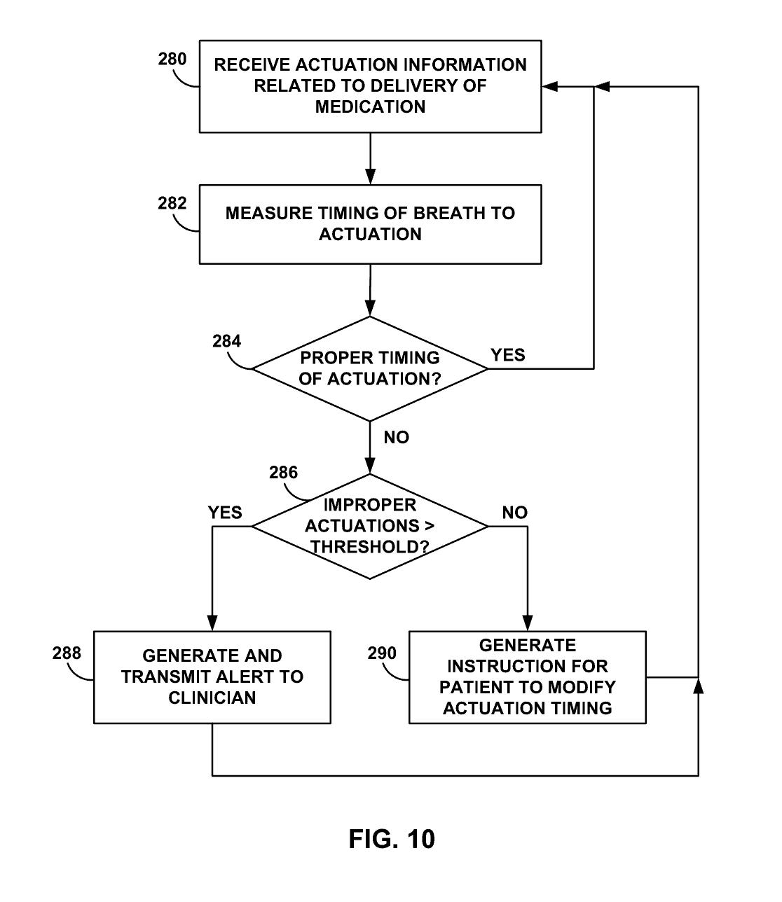

Computing device 22 may be any computing device configured to communicate with PMDD, generate and deliver an alert to instruct patient 12 to actuate canister 20 of PMDD 14, and/or deliver additional functionality related to the treatment of one or more conditions of patient 12. For example, computing device 22 may be a mobile device (e.g., a smartphone, a tablet computer, a smartwatch, a notebook computer, a digital camera, or a portable medical device) or a stationary workstation or desktop computer. In any case, computing device 22 may be configured to communicate with a communication unit of PMDD 14 using wired or wireless communication protocols. In addition, computing device 22 may be configured to communicate with a networked server, networked repository, or remote device via a network (as shown in FIG. 10). In some examples, computing device 22 may transmit data (e.g., to a network server) and receive an analysis of the data, a command based on the transmitted data, or any other information to offload processing functions to the networked server or utilize additional features of the networked server (e.g., access to additional data stored on the network or additional processing power).

In one example, PMDD 14 may include housing 16 configured to accept medication canister 20 containing a medication, where housing 16 includes a dispensing portion (e.g., mouthpiece 18). PMDD 14 may also include a sensor configured to sense air flow within the dispensing portion, and a processor configured to transmit a signal indicative of the sensed air flow, wherein an alert that instructs a user to actuate a medication canister 20 associated with housing 16 is generated based on the transmitted signal. Computing device 22 or PMDD 14 may generate the alert. The alert may be delivered as a visual, audible, and/or tactile alert to the patient 12. In some examples, computing device 22 and/or PMDD 14 may deliver the alert at the appropriate time for actuation as a training tool for the patient. In other words, the patient can actuate canister 20 and manually monitor if the actuation correlated with the delivered alert. Computing device 22 may also generate indications of the effectiveness of the actuation (e.g., whether the actuation was a good (or proper) actuation or a bad (or improper) actuation) based on the air flow data and actuation timing. Computing device 22 may present this information so the patient can review which actuations were good and which actuations were bad. The generation and presentation of information relating to the effectiveness of medication canister actuation using PMDD 14 may be a training tool for the patient to improve actuation and/or inhalation technique. Therefore, this training tool may result in increased compliance with prescribed medication dosages.

Generally, computing device 22 and/or PMDD 14 may generate and deliver the alert in response to the air flow rates detected in real-time such that the user can actuate medication canister 20 after the inhalation has begun and during the inhalation air flow. For example, the alert may be delivered to prompt the user to actuate canister 20 prior to peak inhalation flow rates and/or during the peak inhalation flow rate. In some examples, computing device 22 and/or PMDD 14 may require detection of an exhalation (e.g., an exhalation exceeding a threshold volume or flow rate) prior to the inhalation in order to trigger the alert. In other words, failure to detect the prior exhalation may indicate that the following inhalation may not be sufficient to receive and/or disperse a full dose of medication.

In addition, computing device 22 or PMDD 14 may incorporate algorithms that generate the alert to actuate canister 20 based on prior inhalation flow profiles from patient 12. For example, prior inhalation flow profiles may be used to calculate average or median times of total inhalation and/or times to the peak flow rate and deliver the alert at that anticipated time after the current inhalation was originally detected. In this manner, computing device 22 and/or PMDD 14 may generate a predictive alert to accommodate delays in flow sensing, processing, and data transmission. In addition, the delivery of the alert may be timed to incorporate patient delay (or patient response time) for identifying the alert and then actuating canister 20. This patient delay may be calculated from prior alert to actuation delays and incorporated into the predictive timing used by computing device 22 and/or PMDD 14 for delivering the alert that instructs the patient to actuate canister 20 during the current inhalation. In some examples, computing device 22 and/or PMDD 14 may deliver the alert when it is predicted that the patient should initially depress the canister 20. In other examples, the alert may also be delivered to indicate the actuation and remain delivered (e.g., maintain a light, sound, or vibration) for the entire time during which the patient should inhale and maintain the inhaled breath (e.g., at least five seconds). An alert may be delivered in response to detection that the patient has held a breath for a predetermined amount of time (e.g., five seconds).

In some examples, computing device 22 may display flow rate data in real-time, or as the patient is inhaling and/or exhaling, so that the patient can see the flow rate changes over time and use the information displayed by computing device 22 to determine when to actuate canister 20. For example, computing device 22 may present a bar graph or pendulum that changes according to the inhalation and/or exhalation flow rate to allow the patient to anticipate the optimal time for actuation. In some examples, computing device 22 may add an indication of the optimal time for actuation with the flow rate information so that the patient can tell how well the actuation was timed.

In another examples, PMDD 14 and/or computing device 22 may determine the optimal time to actuate canister 20 during a patient inhalation breath. The patient maybe instructed to practice breathing into PMDD 14, and PMDD 14 and/or computing device 22 may generate and deliver an indication of the optimal time during the inhalation to actuate the canister. For example, the optimal time may be indicated by a light, a sound, or a tactile output. In some examples, the patient can use this mode to identify the optical timing of an actuation during the inhalation. In other examples, PMDD 14 and/or computing device 22 may monitor the breathing patterns prior to an actuation and, responsive to detecting a breathing pattern appropriate for receiving medication from PMDD 14, PMDD 14 and/or computing device 22 may generate and deliver an alert that instructs the patient when to actuate canister 20 of PMDD 14 during a breath in the breathing pattern.

PMDD 14 and/or computing device 22 may also determine the correct time to deliver an alert to the patient to actuate canister 20 of PMDD 14 and/or when the actuation should be detected. The appropriate timing of actuation to the patient breath may be determined based on one or more of the following aspects: the time from the start of the inhalation to the actuation being within a threshold, the total time of the inhalation that includes the actuation is within a threshold, the total exhalation time immediately prior to the actuation is within a threshold; the total volume of inhalation air is within a threshold, the peak flow rate during inhalation and/or exhalation is greater than a threshold, a derivative of the inhalation flow rate is within a threshold, and/or a derivative of the exhalation flow rate is within a threshold. Using one or more aspects of the above timings, the system may determine whether an actuation was appropriate and/or medication was appropriately delivered to the patient.

The sensor of PMDD 14 may be configured to generate the signal indicative of the sensed air flow. PMDD 14 may also include a second sensor configured to detect actuation of the medication canister 20, and PMDD 14 may include the processor to generate a signal indicative of the detected actuation of the medication canister 20 and transmit the signal indicative of the detected actuation. PMDD 14 may also be configured to transmit the signal indicative of the sensed air flow to a mobile computing device (e.g., computing device 11) in communication with the processor of PMDD 14) and transmit the signal indicative of the detected actuation of the medication canister 20 to the mobile computing device in communication with the processor. The processor of PMDD 14 and/or computing device 22 may be configured to generate the alert that instructs the user to actuate the medication canister 20.

In another example, computing device 22 may include one or more processors configured to receive, from PMDD 14, a signal indicative of sensed air flow within a portion of the PMDD and generate, based on the signal, an alert that instructs a user (e.g., patient 12) to actuate medication canister 20 associated with PMDD 14. Computing device 22 may include one or more output devices (e.g., displays, lights, speakers, haptic feedback devices, etc.) configured to deliver the alert to the user.

The one or more processors of computing device 22 may be configured to receive a signal indicative of a detected actuation of medication canister 20, and computing device 22 may include a memory configured to store a representation of the signal indicative of the detected actuation of medication canister 20. The one or more processors of computing device 22 may also be configured to generate a drug delivery log (e.g., an e-diary of medication delivery) based on one or more received signals indicative of respective detected actuation of medication canister 20 and transmit, from computing device 22, the drug delivery log. Computing device 22 may transmit the drug delivery log to another device such as a networked server or remote clinician device. In some examples, the drug delivery log may include detected actuations with or without flow sensor data, as actuations corresponding in time to inhalations may be determined as medication delivery to the patient. Such a drug delivery log may allow the patient and/or health care professional to track compliance of the user to a prescribed drug dosage in an effort to ensure that the patient is receiving the appropriate amount of medication to manage the patient's condition.

In some examples, computing device 22 may determine (e.g., compute) an attribute of the received flow signal relevant to a disease state, such as peak flow rate, inhalation volume, or any other characteristic derived from the flow and/or pressure signal. Computing device 22 may also determine a dose actuation accuracy, which may indicate the time between the presented alert and the detected actuation of canister 20. The dose actuation accuracy may be used to track effectiveness of drug delivery and/or computing device 22, or another device, to update the timing of the presented alert to improve actuation timing. Any of this determined information related to the use of PMDD 14 may be generated by any of PMDD 14 and/or computing device 22, for example, and stored in a single drug delivery log or separate logs (e.g., a flow attribute log, a drug delivery log, and/or a dose accuracy log). Each of these logs (e.g., stored patient information), may be stored on computing device 22, stored on PMDD 16, stored on another computing device associated with the patient and/or clinician, transmitted to another computing device via a network (e.g., a remote computing device associated with a clinician or hospital), and/or stored on a networked computing device (e.g., a networked server). In some examples, an electronic health record (EHR) system may be a health information technology platform configured to receive data transmitted by PMDD 14 or computing device 22 via a network or directly from the respective device. In other examples, PMDD 14 may include a processor and other electronics configured to perform any of the tasks described herein attributed to computing device 22 (e.g., generating logs, storing patient information, and/or transmitting patient information). Any of PMDD 14, computing device 22, or another networked computing device may output, for display, information indicative of the drug delivery log.

As described herein, the alert may include at least one of a visual cue, an audible cue, and a tactile cue. PMDD 14, computing device 22, and any combination thereof, may include one or more output devices configured to deliver the alert that instructs patient 12 when to actuate medication canister 20. In this manner, an alert may be delivered as one, two, or three or more different cues. For example, computing device 22 may deliver the alert by delivering an audible cue such as a sound or pattern of sounds that instructs patient 12 to actuate canister 20. In other examples, computing device 22 may deliver an audible cue and a visual cue. The visual cue may be a display that presents a word, phrase, sentence, image, animation, or any combination thereof. The visual cue may also be a light or other visual indication device that emits a light or changes the color or pattern of light to deliver the alert. A tactile cue may be a mechanical vibration, electrical stimulus, or any other action delivered by a device, such as a haptic feedback device, that can be felt by patient 12 or another user. In some examples, both computing device 22 and PMDD 14 may deliver one or more cues of an alert. In this manner, PMDD 14 and/or computing device 22 may include one or more output devices configured to deliver one or more respective visual cues, audible cues, or tactile cues.

PMDD 14 is described with regards to a hand-held device that can be applied to the mouth of patient 12. In other examples, PMDD may be configured to be used within a breathing circuit (e.g., manual or automated ventilator) attached to patient 12 or configured to be attached to a tube from which air is inhaled by patient 12. In some examples, PMDD 14 may include a reusable portion configured to attach to the front and/or top of housing 16 (or alternatively formed with housing 16) or the top of canister 20. This reusable portion may include a visual status indicator configured to display information to the user when PMDD 14 is being used by patient 12. In other words, the reusable portion may include the visual status indicator at a location of PMDD 14 from which patient 12 can see the visual status indicator during use (e.g., the top of housing 16 or mounted on top of canister 20).

FIG. 1B is a conceptual drawing illustrating an example system 24 that includes a PMDD 26 and computing device 22 in relation to user 12. System 24 is similar to system 10 of FIG. 1A and may be configured to perform the same functions in one or more examples. In addition, PMDD 26 may be similar to PMDD 14. However, PMDD 26 includes two portions: housing 28 and attachable assembly 30.

Housing 28 may be configured to accept a medication canister 20 or any other reservoir for medication directed to treat pulmonary function disorders. Housing 28 may be an off-the-shelf metered dose inhaler housing typically used by patients to receive medication from container 20. Medication may be dispensed out of medication canister 20 and exit housing 28 via mouthpiece opening 36. Housing 28 may be a single piece or constructed using two or more components. Attachable assembly 30 may be configured of a size and shape that facilitates attachment to housing 28. For example, attachable assembly 30 may be configured to “snap” onto the outside of housing 28 so that attachable assembly 30 is secured to housing 28. In addition, or alternatively, attachable assembly 30 may include (e.g., formed of or attached to) one or more clips that attach to an upper edge of housing 28. In some examples, attachable assembly 30 may utilize adhesives, a hook-and-loop closure system, one or more set screws, or any other fastening device to attach to housing 28.

Attachable assembly 30 may be a device or system with one or more electrical components configured to perform the functions described herein. Attachable assembly 30 may include an exterior housing that houses the one or more electrical components within the exterior housing or supports one or more electrical components on the surface or attached to the exterior housing. Attachable assembly 30 may include one or sensors and/or electronics described herein with relation to a PMDD. For example, attachable assembly 30 may include electronic components such as one or more flow sensors that detect air flow going into housing 28 and/or going out of housing 28 from patient breaths. The air flow sensor (not shown in FIG. 1B) may be located within channel 31 behind attachable assembly 30 and within housing 28. Channel 31 may be a continuous flow channel that extends from an inlet between attachable assembly 30 and canister 20, through housing 28, and out of mouthpiece opening 36. In this manner, a portion of housing 28 may be positioned between a portion of attachable assembly 30 and the air flow sensor such that the air flow sensor can sense air flow within housing 30.

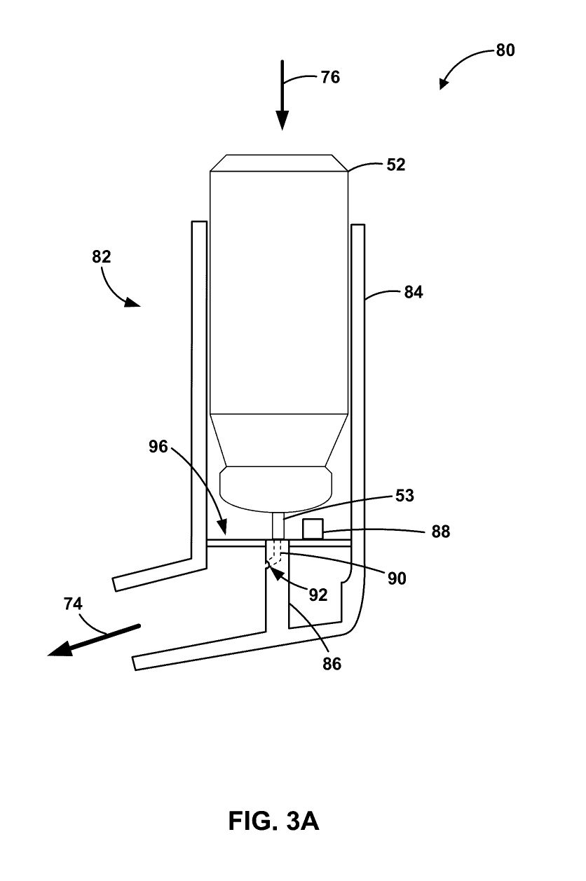

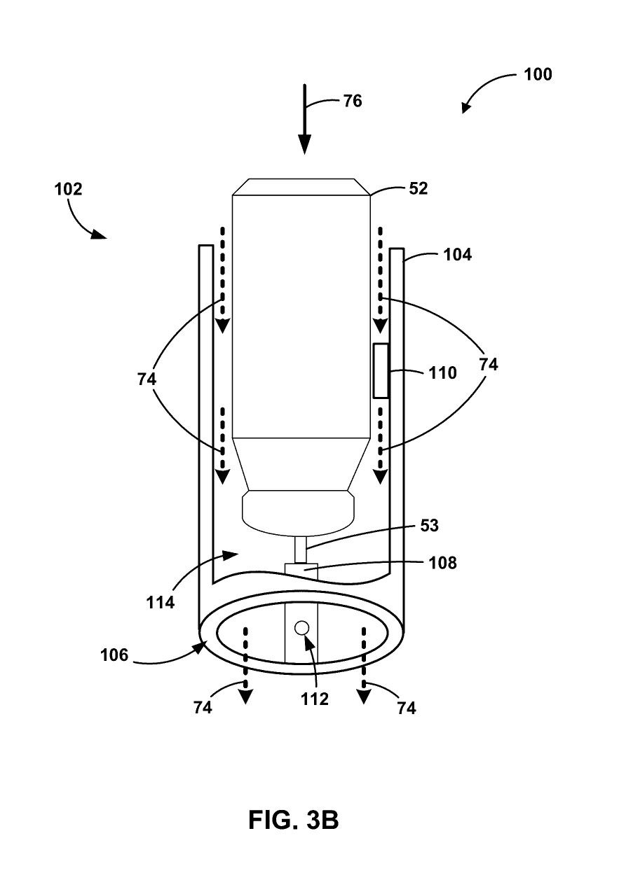

Attachable assembly 30 may also include other sensors such as an accelerometer, electrical switch, temperature sensor, electrodes and corresponding electrocardiogram circuitry, or any other sensors. For example, attachable assembly 30 may include an actuation sensor that detects actuation of canister 20. An actuation sensor may an optical sensor directed at canister 20. The optical sensor may be paired with a high contrast pattern on canister 20 or other mechanism such that the optical sensor can detect movement of canister 20. Attachable assembly 30 may include the actuation sensor adjacent to or otherwise near the airflow sensor outside of or within housing 28. An actuation sensor may alternatively be similar to sensor 88 of FIG. 3A. In addition, attachable assembly 30 may include electronics necessary to operate the one or more sensors, transmit and/or receive data to and from computing device 22 or other devices, and a power source.

Attachable assembly 30 may include one or more output devices configured to deliver an alert to the patient, such as a visual indicator 35 located near the top of the attachable assembly that is visible to the patient during use of PMDD 26. Visual indicator 35 may be a light that turns on, changes brightness, and/or changes color to indicate functions of attachable assembly 30. For example, the processor of attachable assembly 30 may control visual indicator 35 to illuminate in response to an inhalation detected by the flow sensor and/or in response to an actuation of medication canister 20 detected by the actuation sensor. In some examples, visual indicator 35 may provide different colors to indicate that respective events were detected, such as green light for a detected breath and a red light for a detected actuation of canister 20. In another example, visual indicator 35 may indicate when attachable assembly 30 and/or computing device 22 determines that actuation of canister 20 was timed correctly with a detected inhalation from the patient. Visual indicator 35 may illuminate when a correct actuation was determined or visual indicator 35 may present a color representative of a proper actuation (e.g., green) and an improper actuation (e.g., red). Other output devices may include audio devices, tactile devices, displays, or any other components.

Although attachable assembly 30 is shown as attached to the top, or intake, portion of housing 28, attachable assembly 30 may be configured to attach to a different portion of housing 28 in other examples. For example, attachable assembly 30 may attach to a middle portion of housing 28 with a sensor external to housing 28 or a sensor that is inserted through a hole in housing 28. In other examples, attachable assembly 30 may be configured as a mouthpiece such that the patient lips make contact with attachable assembly 30 instead of the mouthpiece portion of housing 28.

FIG. 1C is a conceptual drawing illustrating PMDD 26. FIG. 1C illustrates a rear view of PMDD 26 showing that attachable assembly 30 attaches to the top of housing 28 and does not completely encompass canister 20. Attachable assembly 30 may include side portions that extend along the sides of housing 28 to apply a friction fit attachment between attachable assembly 30 and housing 28. In other examples, attachable assembly 30 may completely encompass the perimeter of housing 28.

FIG. 1D is a conceptual drawing illustrating an example PMDD 32. PMDD 32 may be substantially similar to PMDD 26 of FIG. 1B. However, PMDD 32 may include a single housing 34 with integrated electronics instead of the separate housing 28 and attachable assembly 30 of PMDD 26. In this manner, PMDD 32 may be an alternative pulmonary medication dosing device to PMDD 26 that also includes functionality discussed above with respect to attachable assembly 30. Housing 34 may also be configured to receive canister 20. Medication may be dispensed out of medication canister 20 and exit housing 34 via mouthpiece opening 38, similar to mouthpiece opening 36 of FIG. 1B. PMDD 32 may include visual indicator 39 that is substantially similar to visual indicator 35 of FIG. 1B.

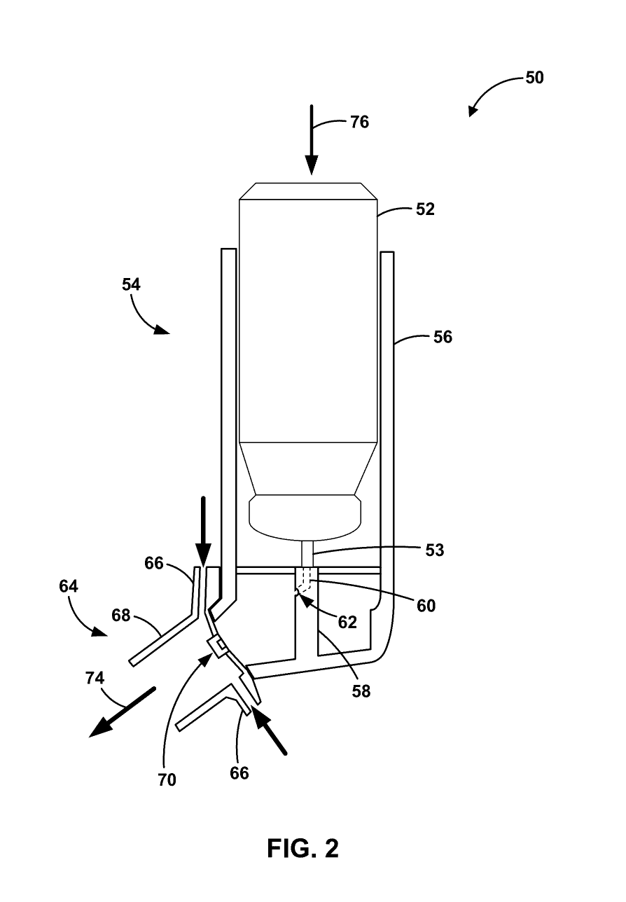

FIG. 2 is a conceptual drawing illustrating an example PMDD 50 that includes a flow sensor. PMDD 50 may be an example of PMDD 14 of FIG. 1A. As shown in FIG. 2, PMDD 50 includes housing 54 coupled to mouthpiece 64. Housing 54 includes canister portion 56 for accepting medication canister 52 and structure 58 that accepts metering valve 53 of canister 52. Structure 58 also defines channel 60 and orifice 62, wherein medication dispensed from canister 52 is directed through channel 60 and out of orifice 62 to patient 12. Without mouthpiece 64, housing 54 may function similar to a manually operated MDI by depressing canister 52 in the direction of arrow 74.

However PMDD 50 also includes mouthpiece 64 attached to housing 64. Mouthpiece 64 may be a delivery portion of PMDD 50 and include functional components such as flow sensor 70 and related electronics. In this manner, mouthpiece 64 may be an add-on device to transform the operation of housing 54 into PMDD 50 configured to detect air flow associated with an inhalation from patient 12. Both housing 54 and mouthpiece 64 may be configured to function together. Sensor 70 may be configured to sense air flow from an inhalation and generate a signal representative of the air flow. Sensor 70 may also be configured to detect medication delivered from canister 52. In other examples, an additional sensor may be configured to detect medication delivered from canister 52 instead of sensor 70. Sensor 70 may detect air flow, pressure, or any other aspects required to sense inhalation and/or the presence of delivered medication.

Mouthpiece 64 may include a nozzle portion 68 shaped and sized to direct air flow to mouth 13 of patient 12. Mouthpiece 64 may also include sensor 70. In some examples, mouthpiece 64 may house one or more circuits required for the function of PMDD 50. In other examples, housing 54 or another add-on assembly configured to attach to housing 54 may include one or more circuits required to perform the functions described herein. Intake portions 66 of mouthpiece 64 define respective intake openings in mouthpiece 64. Intake portions 66 direct air from outside of PMDD 50, toward the location of sensor 70 and the associated sensor, and out of nozzle portion 68 to mouth 13 of patient 12. Although two intake openings are shown in mouthpiece 64, mouthpiece 64 may alternatively be constructed to define only one intake portion or more than two openings. The cross-sectional area of the intake openings may be sized to facilitate appropriate air flow rates through mouthpiece 64. In some examples, mouthpieces with smaller intake openings may be constructed from younger patients in comparison with larger openings for adults. In addition, or alternatively, to intake portions 66, housing 54 may define or more intake openings for drawing air through a portion of housing 54 behind the location of sensor 70.

Sensor 70 may be positioned near the axis running through the center of nozzle portion 68. Although sensor 70 may be positioned at any location within or near mouthpiece 64, a generally center position of sensor 70 may increase the amount of medication distributed to the air stream. Sensor 70 may sense a change in pressure to detect inhalation (e.g., detect a decrease in pressure). Sensor 70, or another sensor, may detect medication delivery from a change in pressure within mouthpiece 64 or housing 56 (e.g., a chamber downstream of orifice 62). For example, the sensor may detect medication delivery by detecting an increase in pressure resulting from the expulsion of medication from canister 52 and orifice 62. This pressure detection method may generate a signal than changes over time to indicate a pressure curve. Pressure exceeding a threshold, or relative threshold from an initial portion of the pressure curve, may be indicative of medication delivery. In other examples, sensor 70 may be attached and adjacent to an inner surface of mouthpiece 64 such that sensor 70 is out of the direct path of medication delivery but still within an area from which inhaled air and/or dispensed medication can be detected. In some examples, sensor 70 may be within a protected air channel that receives a portion of inhaled and exhaled air without being exposed to medication delivered from canister 52.

Mouthpiece 64 may also include an electronics package configured to sense air flow, control sensor 70, and/or transmit data to a computing device that generates and/or delivers an alert to patient 12. The electronics package may also include a power source, such as a rechargeable battery or non-rechargeable battery. The electronics may be disposed inside of mouthpiece 64 and/or on an external surface of mouthpiece 64.

Mouthpiece 64 may be removably attached to housing 54. In other words, mouthpiece 64 may be added to housing 54 when needed, removed from housing 54, reattached to housing 54, or attached to a different housing. Mouthpiece 64 may be attached to housing 54 using a friction fit, an adhesive, fastening devices, indent/detent connections, helical fittings, or any other mechanism. Mouthpiece 64 may be constructed of a rigid, flexible, elastic, transparent, clear, and/or opaque material. In some examples, mouthpiece 64 may be constructed of several different materials, each material directed to a specific feature or as different layers of mouthpiece 64.

In other examples, PMDD 50 may include one or more auxiliary flow channels to sense air flow and/or drug delivery. An auxiliary flow channel may be a tube or other structure configured to run parallel to, and at least partially isolate from a main fluid flow path, and one or more flow sensors (e.g., sensor 70) may be located within the auxiliary flow channel to sense fluid flow. Each of the one or more auxiliary flow channels may be attached within or formed within mouthpiece 68 and/or housing 56. A portion of inhaled air may thus flow through the main fluid flow path and another portion of inhaled air may flow through an auxiliary flow channel. One or more of the auxiliary flow channels may be configured to allow passage of inhaled air and/or dispensed drug from canister 52.

In one example, an auxiliary flow channel may be contained within a housing for the main fluid flow path with fluid communication to the main fluid flow path on both ends of the auxiliary flow channel. In another example, the auxiliary flow channel may merge with the main fluid flow path at an end of the main fluid flow path proximal to the patient while one or more independent inlets to the auxiliary flow channel distal to the patient (or proximal to canister 52). One or more of the independent inlets, or all in some examples, may be isolated from the flow of drug such that drug from canister 52 does not enter the auxiliary flow channel. In some examples, the diameter of one or more auxiliary flow channels may have a diameter and/or cross-sectional shape configured to reduce an associated Reynolds number (e.g., reduce flow turbulence). Lower turbulence within an auxiliary flow channel may improve the signal to noise ratio for the flow sensor within the auxiliary flow channel.

In some examples, multiple auxiliary flow channels may be positioned around and/or within the main fluid flow path through which inhaled air flows. Multiple auxiliary flow channels may allow PMDD 50 to detect inhalation and/or drug delivery regardless of variations in patient use of PMDD 50 (e.g., incomplete seal of the mouth around mouthpiece 64 or angled positioning of mouthpiece 64). If multiple auxiliary flow channels are implemented, each channel may include a respective flow sensor (each sensor may be identical or different from each other). Alternatively, multiple auxiliary flow channels may merge to or split away from a single flow sensor.

FIG. 3A is a conceptual drawing illustrating an example PMDD 80 that includes a canister actuation sensor 88. PMDD 80 may be substantially similar to PMDD 50 of FIG. 2. However, PMDD 80 is constructed with canister actuation sensor 88 to detect the actuation of canister 52 associated with PMDD 80 and housing 82. Actuation sensor 88 may be a pressure switch that generates a signal when canister 52 is depressed in the direction of arrow 76 from patient 12 or another user. Actuation sensor 88 may then detect when medication is released as a result of depressing and actuating canister 52. Sensor 88 may be configured as any type of sensor (e.g., pressure, optical, impedance, temperature, magnetic, electrical switch were an electrical circuit is completed or broken with canister actuation, etc.) that detects the depression or contact of canister 52. In other examples, PMDD 80 may include a sensor configured to detect heart rate (e.g., via an electrocardiogram signal), respiratory rate or blood pressure, or other sensors (e.g. temperature, lung impedance). A temperature sensor may be disposed on housing 82 where a hand contacts housing 84 or on a mouthpiece portion of housing 82 where the patient lips or tongue may contact the temperature sensor. Lung impedance sensors may be utilized to detect pulmonary edema, for example. In this manner, sensor 88 may be an electromechanical switch configured to detect motion between canister 52 and housing 82. Housing 82 may be substantially similar to a generic metered dose inhaler housing, to which sensor 88 and associated electronics may be added to detect canister actuation.

As shown in FIG. 3A, PMDD 80 includes housing 82 coupled to mouthpiece 100. Housing 82 includes canister portion 84 for accepting medication canister 52 and structure 86 that accepts metering valve 53 of canister 52. Structure 86 also defines channel 90 and orifice 92, wherein medication dispensed from canister 52 is directed through channel 90 and out of orifice 92. PMDD 80 also includes actuation sensor 88 coupled to structure 86 and configured to detect actuation or depression of metering valve 53 of canister 52. In some examples, actuation sensor 88 may alternatively be coupled to the top or side of canister 52 instead of attached to housing 82 or otherwise attached to a component of PMDD 80 different than housing 82. PMDD 80 may also include a sensor to detect inhalation, such as sensor 70 of FIG. 2. Computing device 22 may receive data from actuation sensor 88 regarding actuations and use that data to monitor the usage of canister 52 and the number of actuations remaining within canister 52. Computing device 22 may alert the patient when fewer than a threshold number of doses are estimated to remain within canister 52.

FIG. 3B is a conceptual drawing illustrating an example PMDD 100 that includes a flow sensor 110 positioned within the PMDD housing FIG. 3B is a front view of PMDD 100 looking directly into mouthpiece portion 106. PMDD 100 may be substantially similar to PMDD 50 of FIG. 2. However, PMDD 100 is constructed with flow sensor 110 (which may also include canister actuation sensor 88 of FIG. 3A in some examples) to detect air flow from the patient during inhalations (such as during an actuation of medication from canister 52) and/or exhalations. Flow sensor 110 may be positioned within an air channel along the side of canister 52 and within the sides of canister portion 104 of housing 102. Alternatively, flow sensor 110 may be positioned within a central air channel. Air may flow into canister portion 104 in the direction of arrows 74 during inhalation and out of mouthpiece portion 106 via arrows 74. In this manner, flow sensor 110 may detect the flow rate and/or flow speed of air due to an inhalation and/or exhalation from the patient.

The patient may depress canister 52 during an inhalation event to create air flow in the direction of arrows 76. While air flows in the direction of arrows 76 during the inhalation, the patient may depress canister 52 that causes valve 53 to open and expel medication out of valve 53, into structure 108, and out of orifice 112. The medication expelled from orifice is dispensed into the air flow of arrows 74 for distribution into the lungs of the patient. Internal portion 114 within canister portion 104 is shown via a cut away from the external portion of housing 102 for illustration purposes of FIG. 3B.

Flow sensor 110 can be any type of flow sensor described herein or configured to sense the flow of air. In some examples, two or more flow sensors may be positioned within housing 102, such as within canister portion 104. For example, a second flow sensor may be disposed opposite of flow sensor 110 within canister portion 104 (e.g., within any air channel) to provide additional flow data and/or a redundant sensor. In other examples, one flow sensor may detect flow in an inhalation direction of arrows 74 and a second flow sensor may be configured to detect flow in the exhalation direction opposite of arrows 74. In some examples, flow sensor 110 may be reusable for different PMDDs or disposable along with the PMDD. Electronic components may be disposed within housing 102 or attached to the external portion of housing 102, for example. In some examples, mouthpiece portion 106 may house one or more circuits required for the function of PMDD 100.

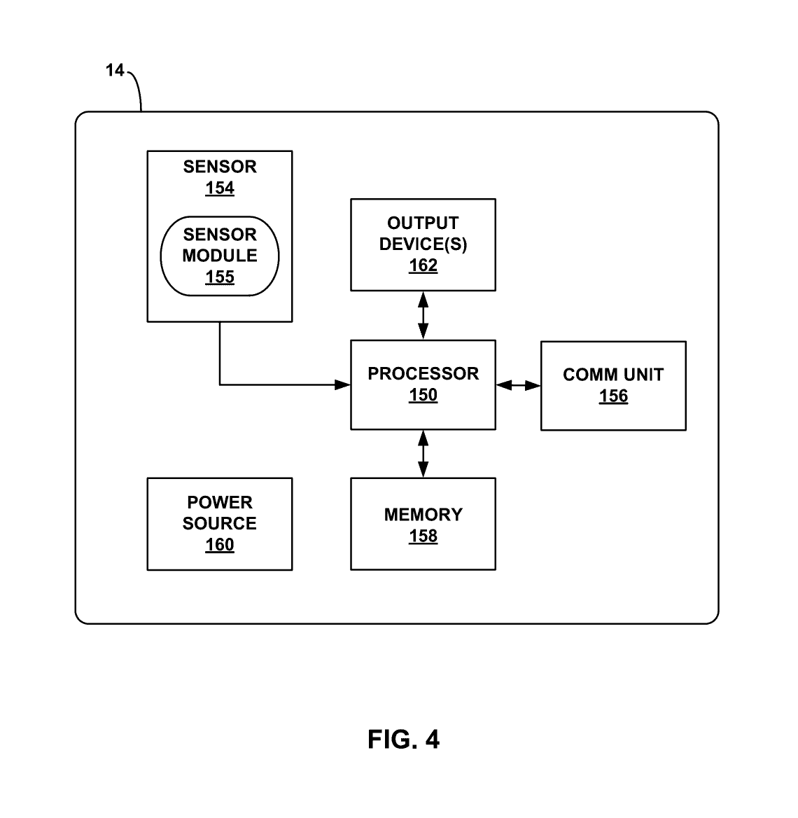

FIG. 4 is a functional block diagram illustrating an example configuration of PMDD 14 of FIG. 1A. In the illustrated example, PMDD 14 includes a processor 150, memory 158, sensor 154, communication unit 156, and power source 160. Memory 158 may be a storage device that includes computer-readable instructions that, when executed by processor 150, cause PMDD 14 and processor 150 to perform various functions attributed to PMDD 14 and processor 150 herein (e.g., generating of a signal indicative of air flow and/or actuation of a medication canister). Memory 158 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 150 may include any one or more of a microprocessor, a controller, a digital signal processor (DSP), an application specific integrated circuit (ASIC), a field-programmable gate array (FPGA), or equivalent discrete or analog logic circuitry. In some examples, processor 150 may include multiple components, such as any combination of one or more microprocessors, one or more controllers, one or more DSPs, one or more ASICs, or one or more FPGAs, as well as other discrete or integrated logic circuitry. The functions attributed to processor 150 herein may be embodied as software, firmware, hardware or any combination thereof.

Processor 150 may receive a signal indicative of air flow from sensor 154 and/or a parameter value representative of the air flow. Processor 150 may store any sensed data in memory 158. Processor 150 may also store indications of sensed air flow operation, communication logs, or any other data related to the use of PMDD 14. Processor 150 may control communication unit 156 to transmit the sensor signal and/or the parameter value to computing device 22 such that computing device 22 can generate/or deliver the alert to patient 12 that indicates when to actuate the medication canister. Processor 150 may also control communication unit 156 to transmit the sensor signal indicative of medication delivery or actuation of the canister.

Memory 158 may be configured to store a variety of operational parameters, therapy parameters, sensed and detected data, and any other information related to the monitoring, therapy and treatment of patient 12. Memory 158 may store, for example, instructions for interpreting sensor signals, volumes of air inhaled by patient 12, medication delivered to patient 12, or any other operational quantitative data. In some examples, processor 150 may offload some data to computing device 22 for further analysis and/or transmission to a networked server or remote device. In some examples, memory 158 may also store communications transmitted to and/or received from computing device 22.

Sensor 154 may be any electro-magnetic sensor configured to output a signal indicative of air flow within PMDD 14. Example sensors may include rotational flow sensors, bending vane sensors, pressure sensors, or any other sensors. Sensor 154 may generate an electrical signal from mechanical deformations or movement of an element of the sensor. In some examples, sensor module 155 may calibrate the signal generated by sensor 154 to determine one or more parameter values such the velocity, flow rate, or volume of air that was delivered. Each of these parameters may be derived from detection of air flow (e.g., flow rate and volume may be derived from a detect air speed or speed may be derived from detected flow rate). In any case, a single measurement of the air flow may be used herein to derive additional parameters of the air flow such as speed, flow rate, total volume (e.g., flow rate over time), breath frequency, air flow changes, acceleration of air flow, etc. PMDD 14 may include one or more additional sensors and/or sensor modules configured to detect different types of events such as medication delivery.

Communication unit 156 includes any suitable hardware, firmware, software or any combination thereof for communicating with another device, such as computing device 22 (FIG. 1A). As described herein, communication unit 156 may transmit sensor signals or parameter values to computing device 22 and/or receive commands generated by computing device based on the sensed signal. Communication unit 156 may be configured to transfer data using any wired or wireless technique, such as Bluetooth, communication according to the 802.11 or Bluetooth specification sets, infrared communication, e.g., according to the IrDA standard, near-field communication, WiFi, or other standard or proprietary telemetry protocols. In some examples, communication unit 156 may support a direct wired connection between PMDD 14 and computing device 22. In one example, a coupling housing may be provided that encases at least a portion of computing device 22 and at least a portion of PMDD 14. The coupling housing may include a wired connection that couples to computing device 22 and PMDD 14 when each device is encased within the coupling housing. The coupling housing may be constructed of a flexible material that allows each of computing device 22 and PMDD 14 to be placed within the coupling housing.

Power source 160 may be any type of device that is configured to hold a charge to operate the circuitry of PMDD 14. Power source 160 may be provided as a rechargeable or non-rechargeable battery. In other examples, power source 160 may also incorporate an energy scavenging system that stores electrical energy from movement of PMDD 14 by patient 12. Power source 160 may be rechargeable via a wired or inductive recharging connection with computing device 22. In some examples, the coupling housing may include a backup battery or other power supply that is configured to recharge power source 160 when PMDD 14 is connected to the power supply of the coupling housing.

In some examples, PMDD 14 may include one or more additional sensors used to monitor therapy and/or other use of PMDD 14. For example, PMDD 14 may include one or more accelerometers that generate data regarding movements of PMDD 14 and/or the position of PMDD 14 with respect to gravity. For example, accelerometer data may be transmitted to a computing device for analysis to determine if the user shook PMDD 14 (and the medication canister 20) prior to medication deliver, if the user shook PMDD 14 for a sufficient duration, if the user shook PMDD 14 with a sufficient magnitude, when actuations occurred, if an acceleration indicates possible damage to the flow sensor. In another example, the accelerometer data may be used to determine if PMDD 14 was oriented correctly (e.g., in an upright position) with respect to gravity such that the medication can exit the canister of PMDD 14 in the appropriate dose. The computing device (e.g., computing device 22) may be configured to alert the user to any problem, suggest actions to resolve the problem (e.g., a reminder to shake PMDD 14 prior to an actuation or an instruction to replace the flow sensor), log the problem, or even transmit an alert regarding the problem to a clinician or clinic.

In some examples, PMDD 14 may include one or more output devices 162 configured to deliver an alert to the user. These output devices 162 may be in addition to, or as an alternative to, computing device 22 delivering an alert. For example, the output devices 162 may include an audio output device (e.g., one or more speakers), a visual indicator device (e.g., one or more lights or displays), and/or a haptic output device (e.g., one or more vibration devices). Each of these output devices 162 may be configured to generate an indication (e.g., an alert) that can be perceived by the user for one or more purposes. Processor 150 may control the output devices to generate and deliver or present the indication to the user. For example, an output device 162 may be configured to deliver an alert related to the optimal time to actuate PMDD 14. As another example, an output device 162 may be configured to output an audible, visual, and/or haptic alert intended to help the user locate a lost PMDD 14. If the user cannot locate PMDD 14, the user can interact with computing device 22 and request computing device 22 to transmit an instruction (via Bluetooth or other wireless communication protocol) for PMDD 14 to deliver a locating alert. In response to receiving the instruction from computing device 22, PMDD 14 may cause an output device 162, such as an audio output device, to deliver an acoustic “beep” or other sound that assists the user in locating PMDD 14. PMDD 14 may additionally, or alternatively, deliver a light and/or vibration from the one or more output devices to assist the user in finding PMDD 14. Computing device 22 may, in response to user input, select which one or more types of alert PMDD 14 will deliver (audible, visual, and/or haptic alert); computing device 22 may specify which type of alert to use in the instructions transmitted to PMDD 14. Once found, the user may terminate the alert by providing an input to computing device 22 such that computing device 22 transmits a termination instruction to PMDD 14 to terminate the alert. The output devices 162 of PMDD 14 may be used to deliver additional alerts to the user, such as alerts to take a dose of medication, alerts regarding PMDD 14 malfunction, or any for any other purpose. Computing device 22 may, in some examples, monitor when to deliver each alert and, in response to determining an alert needs to be delivered by PMDD 14, transmit an instruction to PMDD 14 requesting delivery of the determined alert.

This locator function may also work in reverse between PMDD 14 and computing device 22, where the user can request that PMDD 14 send an instruction to computing device 22 to deliver an output that can help the user locate a lost or misplaced computing device 22. In other examples, different computing devices associated with users other than the patient may be configured to locate computing device 22 in situations in which the patient has lost or misplaced computing device 22.

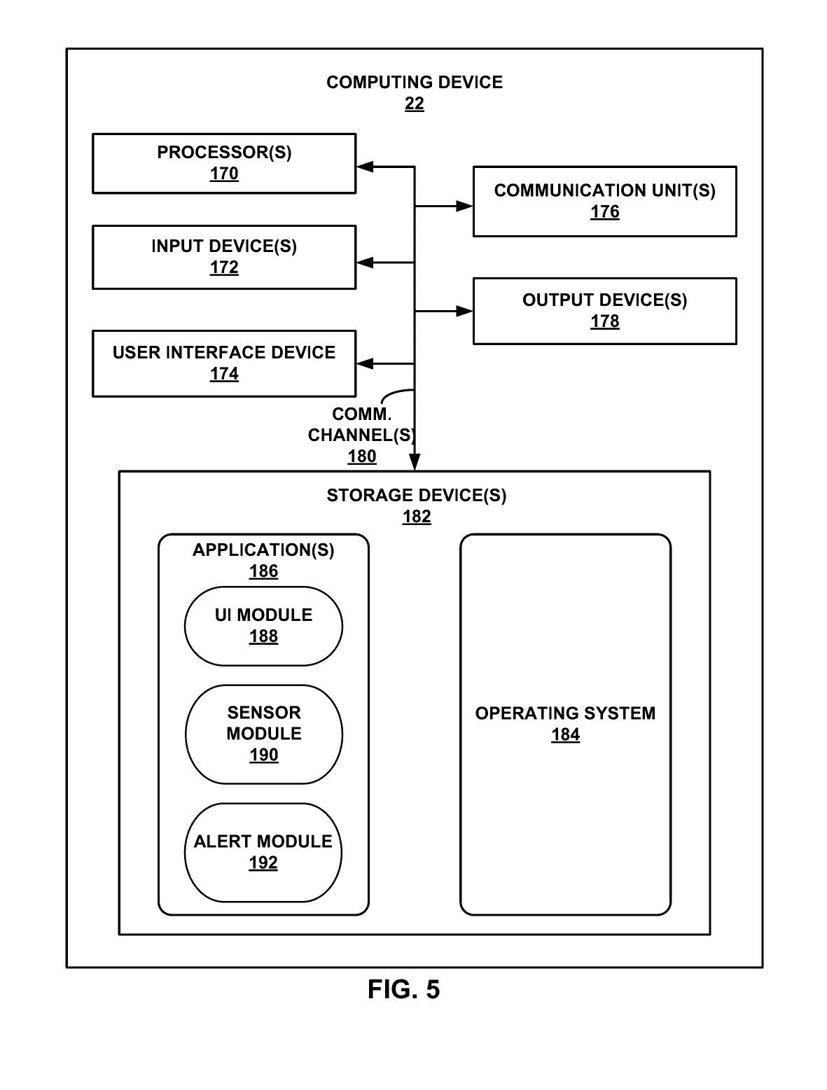

FIG. 5 is a functional block diagram illustrating an example configuration of the computing device 22 of FIG. 1A. Computing device 22 of FIG. 5 is described below within the context of FIG. 1A. In other examples, computing device 22 can include fewer, additional, or different components compared to those illustrated in FIG. 5. For example, although user interface device 174 (“UID 174”) is shown in FIG. 5 as being integral with computing device 22, in other implementations, UID 174 may be operably coupled to computing device 22, e.g., by a wired or wireless data connection. As shown in the example of FIG. 5, computing device 22 includes UID 174, one or more processors 170, one or more input devices 172, one or more communication units 176, one or more output devices 178, and one or more storage devices 182. In this example, storage devices 182 of computing device 22 also include applications 186 (including one or more of UI module 188, sensor module 190, alert module 192) and operating system 184. Communication channels 180 may interconnect each of the components 170, 172, 174, 176, 178, 182, 186, 188, 190, 192, and 184 for inter-component communications (physically, communicatively, and/or operatively). In some examples, communication channels 180 may include a system bus, a network connection, an inter-process communication data structure, or any other method for communicating data.

One or more input devices 172 of computing device 22 may receive input. Examples of input are tactile, audio, and video input. Input devices 172 of computing device 22, in one example, includes a presence-sensitive display, touch-sensitive screen, mouse, keyboard, voice responsive system, video camera, microphone or any other type of device for detecting input from a human or machine. A presence-sensitive display may include both a presence-sensitive input device and a display device. In addition, input devices 172 may include one or more optical sensors, such as a digital camera. The one or more optical sensors may obtain images for detecting air flow within a PMDD and/or counting medication particle delivery. A microphone may also be used to detect inhalations from sound frequency and/or amplitude. In some examples, one or more of input devices 172 may be configured to receive a user input that indicates medication has been delivered and/or the canister has been actuated.

One or more output devices 178 of computing device 22 may generate output. Examples of output are tactile, audio, and video output. Output devices 178 of computing device 22, in one example, includes a presence-sensitive display (which may include a display device), sound card, video graphics adapter card, speaker, cathode ray tube (CRT) monitor, liquid crystal display (LCD), or any other type of device for generating output to a human or machine. As described herein, one or more of output devices 178 may be configured to deliver a user detectable cue of the alert. For example, output devices 178 may be configured to deliver one or more of a visual cue, an audible cue, and a tactile cue. In this manner, patient 12 may receive the alert via one or more of these cues and, in response to receiving the cue, actuate the medication canister.

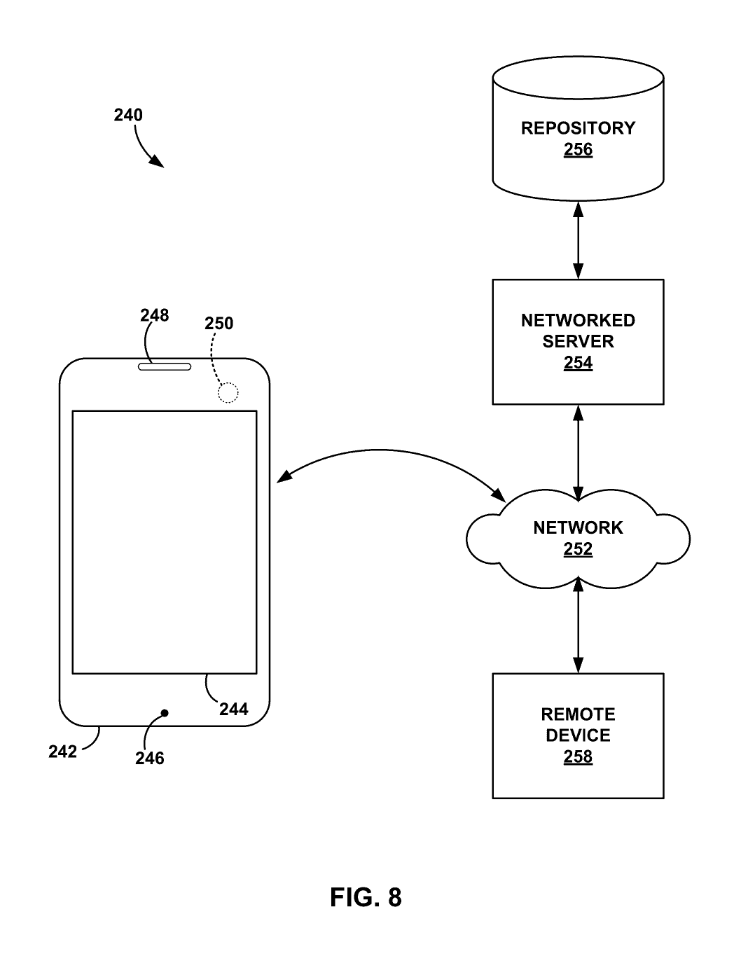

One or more communication units 176 of computing device 22 may communicate with external devices (e.g., a network server such as network server 254 of FIG. 10) via one or more networks (e.g., network 252 of FIG. 8) by transmitting and/or receiving network signals on the one or more networks. For example, computing device 22 may use communication unit 176 to transmit and/or receive radio signals on a radio network such as a cellular radio network. Likewise, communication units 176 may transmit and/or receive satellite signals on a satellite network such as a GPS network. Examples of communication unit 176 include a network interface card (e.g. such as an Ethernet card), an optical transceiver, a radio frequency transceiver, a GPS receiver, or any other type of device that can send and/or receive information. Other examples of communication units 176 may include Bluetooth®, GPS, 3G, 4G, infrared communications, near-field communication modules, and Wi-Fi® radios found in mobile devices as well as Universal Serial Bus (USB) controllers. For example, one or more communication units 176 may be configured to send and/or receive data from one or more wearable devices or other peripheral devices associated with the patient. Such peripheral devices may be configured to provide data regarding patient heart rate, patient movement and/or posture, patient temperature, local air status (e.g., temperature, altitude, or particulate presence), or any other such variable that may affect pulmonary function or other health condition of the patient.

UID 174 of FIG. 5 may include a presence-sensitive display. Computing device 22 may use the presence-sensitive display as an input device and an output device. For example, the presence-sensitive display of UID 174 may include a touchscreen (e.g., a presence-sensitive input device) configured to receive tactile user input from a user of computing device 22. The presence-sensitive display of UID 174 may also include a light emitting diode (LED) display (e.g., a display device) capable of outputting visible information (e.g., a visual cue of the alert) to the user of computing device 22. UID 174 may present a user interface on the presence-sensitive display, which may be related to functionality provided by computing device 22. For example, the presence-sensitive display of UID 174 may present various functions and applications, such as an electronic message client, a map application, an Internet browser for accessing and downloading information from the Internet, and a social media application. In another example, the presence-sensitive display of UID 174 may present a menu of options related to the function and operation of computing device 22, such as screen brightness and other configurable mobile phone settings.

In some examples, the presence-sensitive display may detect an object at and/or near the screen of the presence-sensitive display. As one non-limiting example range, a presence-sensitive display may detect an object, such as a finger or stylus, which is within 2 inches or less of the physical screen of the presence-sensitive display. The presence-sensitive display may determine a location (e.g., an (x,y) coordinate) of the presence-sensitive display at or near which the object was detected. In another non-limiting example range, a presence-sensitive display may detect an object 6 inches or less from the physical screen of the presence-sensitive display, and other exemplary ranges are also possible. The presence-sensitive display may determine the location selected by the object (e.g., user's finger) using capacitive, inductive, and/or optical recognition techniques. In some examples, the presence-sensitive display provides output using tactile, audio, or video stimuli as described with respect to output device 178.

One or more storage devices 182 within computing device 22 may store information required for use during operation of computing device 22. Storage devices 182, in some examples, have the primary purpose of being short term and not long-term computer-readable storage mediums. Storage devices 182 on computing device 22 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art. Storage devices 182 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage devices 182 may store program instructions, data obtained from a PMDD or generated based on PMDD signals, and/or data associated with applications 186, UI module 188, sensor module 190, alert module 192, and operating system 52.

One or more processors 170 may implement functionality and/or execute instructions within computing device 22. For example, processors 170 on computing device 22 may read and execute instructions stored by storage devices 182 that execute the functionality of applications 186, UI module 188, sensor module 190, and alert module 192. These instructions executed by processors 170 may cause computing device 22 to store information within storage devices 182 during program execution, such as notifications, notification objects, and/or information associated applications 186 or any of modules 188, 190, and 192. Processors 170 may execute instructions of applications 186 and/or modules 188, 190, and 192 to perform various functions related to the delivery of medication from a PMDD. For examples, UI module 188 may generate for output visual information related to applications 186. Sensor module 190 may be configured to generate parameter values representative of air flow from received or detected sensor signals and/or medication delivery or actuation of the medication canister. In addition, alert module 192 may be configured to generate alerts that indicate when a user should actuate the medication canister of a PMDD. In other examples, one or more processors 170 may execute instructions of any of applications 186 or modules 188, 190, and 192 to request a network server to perform (or at least partially perform) any of the functions attributed to modules 188, 190, and 192 herein.

Applications 186 may monitor components of computing device 22 to predict and/or detect any operational problems that may prevent the use of computing device 22 with the PMDD to manage patient treatment. For example, applications 186 may monitor the power remaining in a battery that supplies operational power to computing device 22. Applications 186 may generate an alert for display to the user when the battery power drops below a predetermined threshold. The predetermined threshold may be set to allow for a greater duration of remaining power to encourage the patient to maintain sufficient battery charge for use with PMDD 14. For example, the predetermined threshold may be 40 percent or 50 percent of full battery power, as opposed to typical alarms at 10 percent or 15 percent of battery life. In some examples, applications 186 may also or alternatively provide an alert when the battery power of PMDD 14 drops below a predetermined threshold.

One or more applications 186 may also be configured to detect malfunctions with computing device 22 and/or PMDD 14 and/or alert the patient of the malfunction. For example, applications 186 may be configured to detect when there is a problem with communication (e.g., no communication, insufficient bandwidth, or dropped packets of data) between computing device 22 and PMDD 14 and generate an alert for display to the patient. PMDD 14 and/or computing device 22 may also detect malfunctions with a flow sensor or other component of PMDD 14. PMDD 14 may generate and transmit an error signal to computing device 22, and applications 186 may interpret the error signal and generate a corresponding alert for display to the patient. Alternatively, applications 186 may analyze data received from PMDD 14 and determine, based on the analysis, that one or more components of the PMDD 14 are malfunctioning. For example, applications 186 may analyze flow data received from a flow sensor within PMDD 14 and determine, based on the received data, that there is a fault with the flow sensor. Applications 186 may generate an alert that specifies the detected error with PMDD 14 and/or provides one or more troubleshooting instructions for the patient to correct the detected error.

Applications 186 may provide additional functionality for computing device 22 in the management of patient therapy. In one example, applications 186 may utilize an accelerometer or other motion sensor within computing device 22 or communicate with one or more external activity devices (e.g., wearable devices connected to the patient) to monitor activity and exercise of the patient. Applications 186 may track patient activity any generate appropriate alerts for the patient based on the activity. For example, applications 186 may prompt the patient to take a dose of medication prior to or during the activity. In other examples, applications 186 may generate an alert for the patient requesting the patient to stop activity to avoid possible respiratory events. In this manner, applications 186 may facilitate communication of computing device 22 to devices attached to the patient (e.g., wearable devices) such as an electrogram sensing device, a heart rate monitor, a blood pressure monitor, or any other such device. In other examples, applications 186 related to patient therapy may communicate with one or more other applications executing on computing device 22 that track patient information such as an electrocardiogram, heart rate, blood pressure, weight, or any other information.

In some examples, the PMDD (e.g., PMDD 14) may include one or more electrodes configured to detect a heart rate of the patient independently or during a lung function test. Alternatively, computing device 22 may include one or more electrodes for obtaining an electrogram and/or heart rate of the patient or computing device 22 may communicate with a wearable device that obtains heart rate information. In other examples, a coupling housing that combines PMDD 14 and computing device 22 may incorporate electrodes for monitoring patient heart rate. In some examples, computing device 22 may utilize the obtained heart rate to detect tachycardia or other indication of respiratory distress of the patient. The patient may alternatively use a sensor for a predetermined period of time that detects respiratory rate of the patient. Applications 186 may calculate a respiratory rate based on the obtained information and determine when the patient is in respiratory distress. In response to detecting respiratory distress, applications 186 may prompt the patient to call an emergency service for help, take medication, and/or contact a clinician.

Applications 186 of computing device 22 may also provide a coaching feature to aid the patient in achieving proper actuation and improve overall health. Applications 186 may monitor the number of medication doses received by the patient, lung function, and any other metrics and provide suggestions to the patient regarding when to take a dose of medication. Applications 186 may also monitor actuation timing and provide, based on the actuation timing, suggestions to the patient regarding whether the actuations of canister 52 are too early or too late in the breath and/or whether the patient should modify the inhalations during actuations. In addition, applications 186 may monitor patient weight, eating habits, exercise habits, sleep patterns, and any other habits that may affect respiratory health such as asthma or chronic obstructive pulmonary disease (COPD). Applications 186 may utilize clinician inputs or specifications when executing the coach feature.

In some examples, applications 186 may provide a goal feature that sets various goals for certain periods of time, such as daily, weekly, monthly, or overall treatment goals. For example, applications 186 may set lung function goals, a maximum number of asthma attacks during the period of time, an activity goal for the period of time, a number of successful actuations of PMDD 14, or any other goals related to therapy. Lung function goals may include peak expiratory flow targets and/or forced expiratory volume targets.

Although the components of computing device 22 are illustrated in the example of FIG. 5 as within a common housing, one or more components may instead be wired or wirelessly tethered to computing device 22. For example, output device 178 (e.g., a display device) may be physically separate from computing device 22. In other examples, an optical sensor may not reside within a housing of computing device 22.

Computing device 22 may also store data related to the use of PMDD 14 in storage devices 182. For example, computing device 22 may store information or data related to the use of PMDD 14 (e.g., delivery of medication from the medication canister). For example, stored data may include information indicating the time of each delivery of medication or actuation of the medication canister, an indication of whether medication was delivered properly in conjunction with an inhalation, the frequency with which medication was delivered, the amount of medication delivered to the patient, the amount of medication remaining in a canister, the number of prescription refills remaining in an account of patient 12, changes in frequency with which patient 12 requests medication, inhalation volume or flow, environmental condition information such as location, air quality, or allergens present when an actuation was detected, pulmonary function data captured for the patient, or any other information related to the use of PMDD 14 and/or computing device 22. Computing device 22 may transmit this information to a clinician device (e.g., remote device 258 of FIG. 8) or a server (networked server 254 of FIG. 8) periodically, in response to generation of the data, and/or in response to a request for the information.

As discussed above, computing device 22 may be used to communicate with PMDD 14 to instruct PMDD 14 to deliver an alert to aid the user in finding a lost or misplaced PMDD 14. In addition, computing device 22 may provide functionality that aids the patient in determining when to bring PMDD 14 with the patient. For example, computing device 22 may utilize a calendar or other time function that tracks typical times in which the patient goes to work or otherwise leaves the house. In this manner, in advance of the patient leaving the house, computing device 22 may generate and/or deliver a reminder for presentation to the user that reminds the patient to bring PMDD 14 with them. Computing device 22 may additionally or alternatively track the geo-location (e.g., via global positioning system) of computing device 22 (and by proxy, the patient). If the patient leaves the house, computing device 22 may detect that the geo-location of computing device 22 has changed and generate a reminder for the patient to bring PMDD 14. For example, computing device 22 may display a message that reads “Did you have your inhaler?” Computing device 22 may allow the patient to clear the message. In some examples, computing device 22 may only deliver the reminder in response to determining that the patient is leaving the house and that computing device 22 does not detect the presence of PMDD 14. In other words, computing device 22 may determine that a Bluetooth connection with PMDD 14 has been broken or a location request transmitted (via Bluetooth or any other short-range wireless communication) from computing device 22 to PMDD 14 has not been answered by PMDD 14.

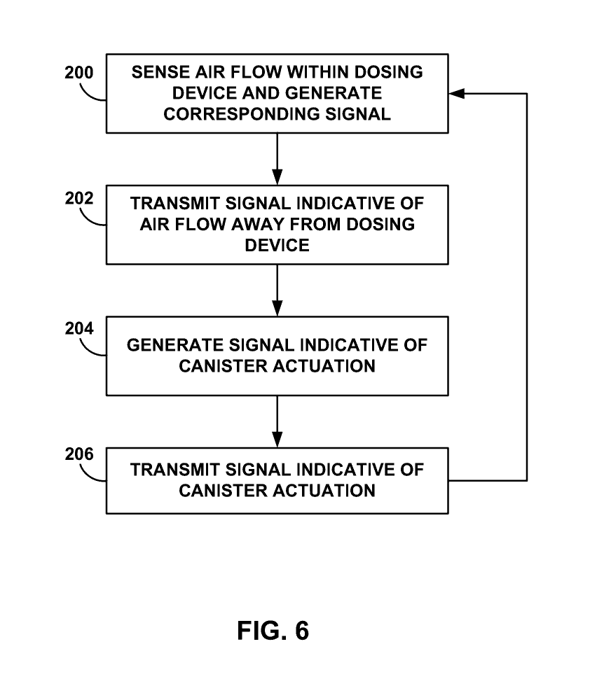

FIG. 6 is a flow diagram of an example process for detecting air flow and canister actuation associated with a PMDD. The example processor of FIG. 6 is described with respect to components (e.g., processor 150 and sensor 154) of PMDD 14 of FIGS. 1 and 6.

However, any of PMDDs 14, 50, 80, and 140 may perform the process of FIG. 6 in other examples. In some examples, one or more steps of the process of FIG. 6 may be performed by a different computing device (e.g., computing device 22), and PMDD 14 may also perform one or more functions attributed to computing device 22.

As shown in FIG. 6, processor 150 may control sensor 154 to sense the air flow within PMDD 14 and generate a signal indicative of the sensed air flow to be transferred to processor 150 (200). In some examples, sensing air flow may include sensing a velocity of air flow using a flow sensor. In other examples, air flow may be sensed using one or more pressure sensors, where changes in pressure are indicative of magnitude and/or direction of air flow within PMDD 14. In some examples, sensor module 155 may also determine one or more parameter values (e.g., a flow velocity, a flow rate, pressure, etc.) representing the signal.

Processor 150 may control communication unit 156 to transmit the signal indicative of air flow to computing device 22 (202). The signal may thus be indicative of inhalation, and computing device 22 may generate and/or deliver the alert that instructs the patient when to actuate the medication canister during the inhalation. Processor 150 may also generate a signal indicative of canister actuation that indicates medication was delivered (204). Processor 150 may control communication unit 156 to transmit the signal indicative of the canister actuation to computing device 22.

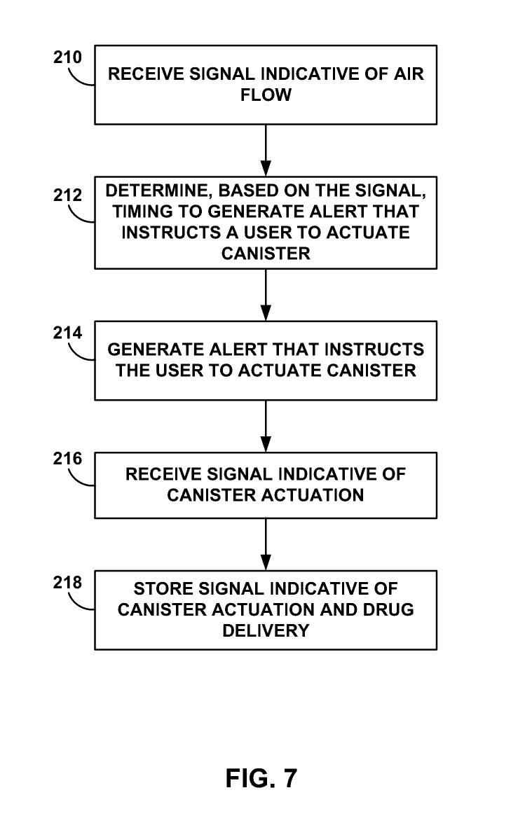

FIG. 7 is a flow diagram of an example process for generating an alert that instructs a user to actuate a PMDD. The example processor of FIG. 7 is described with respect to components (e.g., processor 170, UI device 174, and communication unit 176) of computing device 22 of FIGS. 1 and 7. However, in other examples, one or more aspects of the processor of FIG. 7 may be performed by one of PMDDs 14, 50, 80, and 140 instead of at the different device of computing device 22.

As shown in FIG. 7, processor 170 may receive a signal indicative of air flow resulting from patient inhalation (210). Processor 170 may then determine, based on the signal, timing to generate an alert that instructs a user (e.g., patient 12) to actuate a medication canister associated with PMDD 14 (212). Processor 170 may then generate an alert that instructs the user to actuate the canister (214). Computing device 22 may deliver, via one or more output devices 178, the alert in the form of one or more visual cues, audible cues, and/or tactile cues. Alternatively, processor 170 may control communication unit 176 to transmit the alert to another device, such as PMDD 14, for delivery of the alert in the form of one or more cues.

Processor 170 may also receive, from PMDD 14, a signal indicative of canister actuation or medication delivery (216). Processor 170 may store the signal indicative of canister actuation and drug delivery (218). Alternatively or additionally, one or more of input devices 172 may be configured to receive a user input that indicates medication has been delivered and/or the canister has been actuated. In some examples, processor 170 may generate, or modify, a drug delivery log to include the canister actuation or medication delivery event. Processor 170 may also transmit the drug delivery log or any other event information to another device for review of the use of PMDD 14 and medication delivery.

FIG. 8 is a conceptual diagram of an example system 240 that includes a computing device 242 and a networked server 254 associated with use of PMDD 14. Computing device 242 may be similar to computing device 22 described herein. Although computing device 242 may generate alerts and other information regarding use of PMDD 14, computing device 242 may offload one or more processing tasks to a networked server 254. Networked server 254 may provide additional processing power and/or access to updated clinical instructions related to the use of PMDD 14. For example, networked server 254 may function as an electronic health record system (EHR) for the clinic or hospital associated with the patient. In addition, data obtained by computing device 242 may be transmitted for storage at repository 256 and/or remote device 258. Using remote device 258, a clinician may track the use of a PMDD and the condition of patient 12.

As shown in FIG. 8, system 240 includes computing device 242 (e.g., an example of computing device 22 of FIGS. 1 and 7), network 252, networked server 254, and repository 256. Computing device 242, in some examples, is or is a part of a portable computing device (e.g., a mobile phone, a smartphone, a netbook, a notebook, a tablet device, or a smart watch). In other examples, computing device 242 may be at least a part of a digital camera, a music player, or any other device that a user may carry or move between different locations. Computing device 242 may also connect to network 252 (e.g., a wired or wireless network). Although network 252 may be a single network, network 252 may be representative of two or more networks that allow computing device 242 to communicate with networked server 252.

Computing device 242 may include display device 244, rear camera 250 (e.g., an optical sensor), microphone 246, and speaker 248. Display device 244 may include one or more input devices and/or output devices so that the user can communicate with computing device 242. In one example, display device 244 may include a touch screen interface (e.g., a presence-sensitive display that includes a presence-sensitive input device). In other examples, display device 244 may include a display and one or more buttons, pads, joysticks, mice, tactile device, or any other device capable of turning user actions into electrical signals that control computing device 242. In any example, the user may interact with display device 154 or any other input devices to provide input prior to or during the processes described herein.

Rear camera 250 may enable computing device 242 to capture images (e.g., still images and/or video) of the environment surrounding computing device 242, including detection of a moving element of a PMDD. Rear camera 250 may include one or more optical sensors capable of generating high-resolution images. For example, the optical sensor may include more than one million pixels (a one megapixel sensor), more than five million pixels (a five megapixel sensor), or even more than ten million pixels (a ten megapixel sensor). In some examples, computing device 242 may include two or more cameras disposed on any surface of computing device 242 or coupled to computing device 242 using a cable. Alternatively, rear camera 250 may be placed on the front or other surface of computing device 242.

Microphone 246 may be configured to capture sound around computing device 242, such as user speech, speech from other people, and environmental sounds including inhalations from patient 12. Speaker 248 may be configured to generate and deliver audio to the user such as contact speech or other sounds. In some examples, computing device 242 may include more than one microphone 246 and speaker 248. Although microphone 246 and speaker 248 may be located on or within a housing of computing device 242, microphone 246 and/or speaker 248 may be electrically coupled to computing device 242 via one or more cables. Microphone 246 is an example of an audio input and speaker 248 is an example of an audio output. In other examples, computing device 242 may include additional, or alternative, audio inputs and audio outputs that include a sensor or direct electrical connection configured to accept audio from an attached device or deliver audio to an attached device.

Computing device 242 and networked server 254 may cooperatively function to provide the functionality described herein. Computing device 242 may determine the geographical location at which the computing device is located. For example, computing device 242 may include a device location module, for example, that obtains data from GPS satellites, cellular network access points, or local area network access points, or any other device from which data regarding the position of computing device 242 can be obtained. Computing device 242 and/or networked server 254 may use this geographical location of computing device 242 to suggest when to take a dose of medication based on reported air quality readings, excessive heat, excessive cold, known circumstances certain cities or rural areas, or even air particulates such as pollen or other allergens. These environmental indications may be obtained from a manufacturer of the PMDD, the medication, another company, a clinic or hospital, or a governmental agency. Computing device 242 may then provide an alert to patient 12 to suggest steps to avoid such areas, take a dose of medication to prevent possible symptoms, or otherwise modify medication dosage or therapy. In some examples, the thresholds for each of the environmental conditions or indications may be set by a clinician. For example, a clinician may transmit a list of threshold values for each respective environmental condition to computing device 242, where computing device 242 stores the list of threshold values for use in determining when each environmental condition exceeds the threshold and an alert should be delivered to the patient. The list of thresholds, or at least a portion of the list, may be viewable by the patient. In some examples, the patient may be allowed to adjust one or more of the threshold values. The allowed patient adjustment may be unlimited or only within a predetermined range or predetermined variance from the clinician set value.

In addition, or alternatively, computing device 242 may receive data from one or more peripheral devices (e.g., a wearable device) associated with the patient. The peripheral device may provide data related to physiological status of the patient such as patient temperature, heart rate, respiration rate, activity level, or any other information that may affect whether or not the patient should take a dose of medication or whether the patient should stop such activity. Computing device 242 may determine to generate and deliver an alert in response to any of these inputs, or a combination of several inputs, exceeding respective thresholds. The alert may request that the patient avoid certain areas, change currently detected activity, suggest that the patient take a dose of medication, or provide any other information related to the patient's pulmonary function. As discussed above with respect to the environmental condition thresholds, a clinician may set the threshold values for one or more of the sensed conditions. The patient may or may not be allowed to adjust the threshold values.

Computing device 242 may provide a variety of features utilizing networking over network 252 or with any other remote device. For example, computing device 242 may include an appointment reminder feature which notifies the patient of an upcoming appointment with a physician. Computing device 242 may be connected with the office of the physician and receive push notifications from a networked server associated with the office. In some examples, computing device 242 may receive, from the office server, requests for the patient to make a new appointment. The request from the office server may be generated based on information received from the PMDD and/or computing device 242 related to the use of PMDD and/or lung function of the patient. For example, data transmitted from computing device 242 to the physician's office may be processed by the office server, and the office server may generate a request for a new patient appointment in response to determining that lung function and/or actuation of the PMDD falls below an acceptable threshold.

In some examples, computing device 242 may provide emergency contact shortcuts to clinicians and/or emergency health care. These shortcuts may be provided on a home screen of computing device 242 or via an application specific to management of the patient's pulmonary health care. For example, computing device 242 may provide a single button that, when selected by user input, calls a pulmonologist that manages the patient's care. Computing device 242 may transmit the most recent lung function information and/or actuation information stored in computing device 242 to the physician's computing device in response to initiation of the emergency call via the emergency contact shortcut.

Transmission of information or any other data from computing device 242, may require a connection between computing device 242 and networked server 254 using network 252. Both computing device 242 and network server 254 may connect to network 252. Network 252 may be embodied as one or more of the Internet, a wireless network, a wired network, a cellular network, or a fiber optic network. In other words, network 252 may be any data communication protocol or protocols that facilitate data transfer between two or more devices. Network server 254 may also connect to repository 256 for storing sets of data or retrieving archived data related to patient 12 or the use of PMDD 14. Network server 254 and repository 256 may each include one or more servers or databases, respectively. Network server 254 may include one or more servers, desktop computers, mainframes, minicomputers, or other computing devices capable of executing computer instructions and storing data. In some examples, functions attributable to computing device 242 or computing device 22 herein may be attributed to respective different servers such as networked server 254 for respective functions. Repository 256 may include one or more memories, repositories, hard disks, or any other data storage device. In some examples, repository 256 may be included within network server 254.

Repository 256 may be included in, or described as, cloud storage. Network server 254 may access the cloud and retrieve data as necessary. In some examples, repository 256 may include Relational Database Management System (RDBMS) software. In one example, repository 256 may be a relational database and accessed using a Structured Query Language (SQL) interface that is well known in the art. Repository 256 may alternatively be stored on a separate networked computing device and accessed by network server 254 through a network interface or system bus. Repository 256 may in other examples be an Object Database Management System (ODBMS), Online Analytical Processing (OLAP) database or other suitable data management system. In some examples, repository 256 may be configured to operate as part of an electronic health record system.

As described herein, information may be transmitted between computing device 242 and PMDD 14, for example, using any number of pathways. For example, computing device 242 may be networked to PMDD 14 via a network gateway. In another example, PMDD 14 and computing device 242 may connect to respective gateways that couple to the cloud (e.g., the internet). Clinicians may communicate with computing device 22 and/or PMDD 14 via the cloud and gateways as necessary. In other examples, any devices described herein may be directly connected via wired or direct wireless communication protocols. A clinician may be able to manage applications 186, or settings related to the acquisition of data or delivery of therapy, directly via network 252 or direct communication.

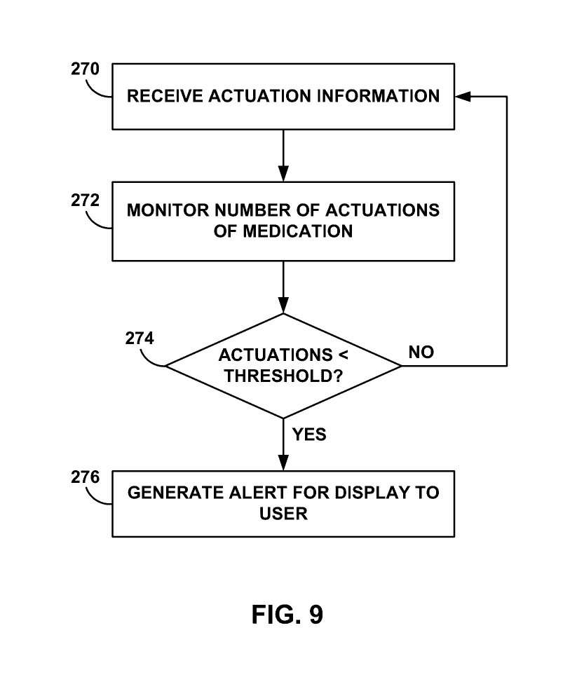

FIG. 9 is a flow diagram of an example process for monitoring the number of medication doses received by a patient. The example processor of FIG. 9 is described with respect to components (e.g., processor 170, UI device 174, and communication unit 176) of computing device 22 of FIGS. 1 and 7 and PMDD 14 (although any other PMDD may be used as well). In some examples, applications 186 may perform these features via processor 170. However, in other examples, one or more aspects of the processor of FIG. 9 may be performed by a different computing device or a PMDD in other examples.

As shown in FIG. 9, processor 170 receives actuation information from PMDD 14 and/or memory of computing device 22 (270). Actuation information may include any data representative of an actuation of a canister within PMDD 14 such as detection of canister actuation or flow information indicative of an actuation. Actuation information may be used to determine when the patient received medication from PMDD 14. Processor 170 may monitor the number of actuations of the medication (272) and determine a metric of patient therapy based on the number of actuations (274). If the number of actuations do not exceed a predetermined threshold (“NO” branch of block 274), processor 170 may continue to receive actuation information (270). If the number of actuations exceed the predetermined threshold (“YES” branch of block 274), processor 170 may generate an alert for display to a user (276). The user may be the patient associated with computing device 22 and PMDD 14 in one example. In addition or alternatively, the user may be the clinician monitoring the patient associated with PMDD 14 in another example.

In some examples, the number of actuations may be the number of actual actuations detected from PMDD within a certain period of time. In other examples, the number of actuations may be the number of doses of medication delivered to the patient from PMDD 14. Alternatively, the number of actuations used in the process of FIG. 9 may only the number of proper (or effective) doses of medication delivered to the patient. In addition, the threshold of decision block 274 may relate to an appropriate threshold. The threshold may be the number of actuations within the previous N seconds (i.e., within a predetermined period of time), within the current calendar day, since the last lung function test was performed within PMDD 14, since the patient performed an activity, or since the last environmental condition threshold has been exceeded. In this manner, the monitoring the number of actuations may allow computing device 22 to monitor the patient's use of medication over time. The use of medication may be used as a factor in determining when to deliver, by computing device 22, a request for the patient to take another dose of medication and/or transmit an alert to a clinician. In this manner, computing device 22 may track patient compliance with a prescribed treatment and alert the patient when medication has not been taken at the scheduled time or within a defined amount of time.

FIG. 10 is a flow diagram of an example process for monitoring the timing of actuations used to deliver medication doses to a patient. This example process may be referred to as a training tool to improve the medication delivery using PMDD 14, for example, by the patent. The example process of FIG. 10 is described with respect to components (e.g., processor 170, UI device 174, and communication unit 176) of computing device 22 of FIGS. 1 and 7 and PMDD 14 (although any other PMDD may be used). In some examples, applications 186 may perform these features via processor 170. However, in other examples, one or more aspects of the processor of FIG. 10 may be performed by a different computing device or a PMDD in other examples.

As shown in FIG. 10, processor 170 receives actuation information related to the delivery of medication from PMDD 14 (280). The actuation information may include data representative of air flow from patient inhalation and an indication of actuation of the medication canister within PMDD 14 that delivered the medication dose. Processor 170 may measure the timing of the patient breath (i.e., inhalation) with or without relation to the actuation of the canister (282). Measurement of the timing of the patient breath and the actuation of the canister (i.e., the delivery of the medication) may be useful in determining whether or not the patient is receiving the optimal amount of medication to the lungs. If actuation occurs too early in the breath, or too late in the breath, the patient may not receive the full dose of medication intended from the actuation. Processor 170 may utilize an algorithm to determine whether or not the actuation was properly timed by the patient in relation to the inhalation (e.g., whether the actuation was effective at delivering the medication into the air flow). If processor 170 determines that proper timing was present between the inhalation and the actuation (“YES” branch of block 284), processor 170 may continue to receive additional actuation information (280).

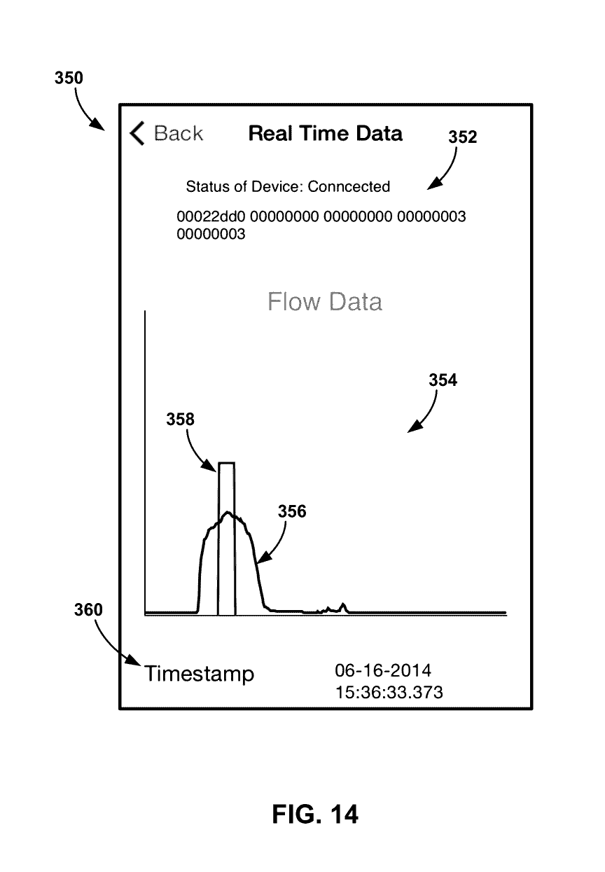

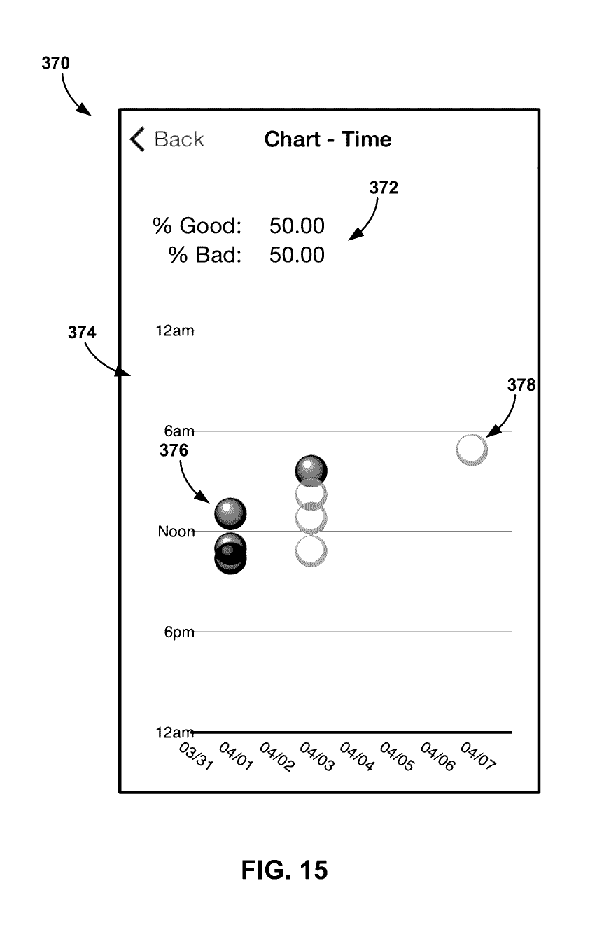

If processor 170 determines that the timing of the actuation was improper (“NO” branch of block 284), processor may then determine if the number of improper actuations that have occurred exceed a predetermined threshold (286). If the number of improper actuations do not exceed the threshold (“NO” branch of block 286), processor 170 may generate an instruction for presentation to the patient to modify the actuation timing (290). In some examples, the instruction may indicate whether the patient needs to actuate the canister earlier in the breath, later in the breath, or how to modify the inhalation breath taken by the patient. In this manner, the generated instructions may be provided as part of a training tool to improve the medication delivery technique of the patient. If processor 170 determines that the number of improper actuations exceed the threshold (“YES” branch of block 286), processor 170 may generate and transmit an alert to a clinician indicating that the improper actuations from the patient exceeds the threshold (288). In some examples, processor 170 may generate the alert for the clinician (288) and generate an instruction for the patient (290) if the number of improper actuations exceeds the threshold (286). An example user interface that may relate such instructions is shown in the example user interfaces 350 of FIG. 14 and 370 of FIG. 15.

The timing of the actuation may be measured using different methods. In one example, processor 170 may determine appropriate timing based only on flow data. For example, processor 170 may determine that the timing was proper (e.g., actuation was effective at delivering medication) if the total volume of the inhalation is greater than a minimum volume and less than a maximum volume, the flow rate was greater than a minimum flow rate and less than a maximum flow rate, and/or the rate of change in flow rate is greater than a minimum flow rate change and less than a maximum flow rate change. In other examples, processor 170 may determine that the actuation timing was proper when the actuation occurred within a predetermined time of a maximum flow rate, a flow rate initially exceeding a flow rate threshold, and/or the actuation occurring within a predetermined period of time following the initiation of the inhalation. Other thresholds and metrics may also be employed to determine the proper timing of the actuation.

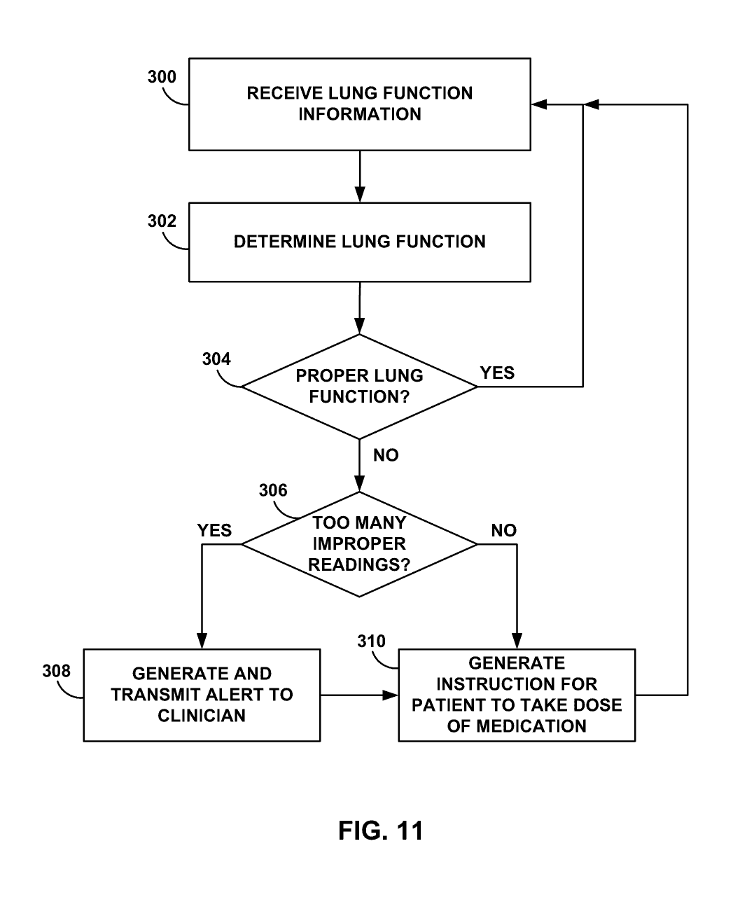

FIG. 11 is a flow diagram of an example process for monitoring lung function of a patient. The example processor of FIG. 11 is described with respect to components (e.g., processor 170, UI device 174, and communication unit 176) of computing device 22 of FIGS. 1 and 7 and PMDD 14 (although any other PMDD may be used). In some examples, applications 186 may perform these features via processor 170. However, in other examples, one or more aspects of the processor of FIG. 11 may be performed by a different computing device or a PMDD.

As shown in FIG. 11, processor 170 receives lung function information related to the patient from PMDD 14 (300). The lung function information may include data representative of air flow from patient inhalation and/or exhalation. For example, processor 170 may calculate peak flow rates and/or total volume of an inhalation or exhalation. This data may be collected during the delivery of medication and/or during a lung function test separate from delivery of medication. Computing device 22 may prompt the patient to perform a lung function test prior to medication delivery, after medication delivery, and/or as a part of drug delivery. In this manner, computing device 22 may prompt the user to regularly check lung function. Processor 170 may, based on the received lung function information, determine the lung function of the patient at a given time (302). Processor 170 may utilize an algorithm to determine whether or not the lung function is proper, such as using one or more thresholds and/or analyzing any changes between different inhalations/exhalations over time. A clinician may set the algorithm and/or thresholds, such as acceptable levels of peak flow rates. Example lung function parameters may include peak expiratory flow rate, forced expiratory volume (FEV) (e.g., forced expiratory volume in 1 second (FEV1)), inhalation volume, or any other respiratory measurement. If processor 170 determines that the patient's lung function is proper, (“YES” branch of block 304), processor 170 may continue to receive additional lung function information (300).

If processor 170 determines that the lung function was improper (“NO” branch of block 304), processor 170 may then determine if the number of improper readings of lung function exceed a predetermined threshold (306). If the number of improper lung function tests do not exceed the threshold (“NO” branch of block 306), processor 170 may generate an instruction for presentation to the patient to take a dose of medication as therapy for the improper lung function (310). For example, if the peak flow rate does not exceed a satisfactory threshold, processor 170 may instruct the patient to deliver a dose of medication. In some examples, the instruction may indicate when to take the next dose of medication or how many doses of medication to take. If processor 170 determines that the number of improper lung function tests exceed the threshold (“YES” branch of block 306), processor 170 may generate and transmit an alert to a clinician indicating that the number of improper lung function tests from the patient exceeds the threshold (308). In other words, computing device 22 may alert a clinician in response to a significant decline in pulmonary function. Computing device 22 may also alert the patient of diminished lung function. Processor 170 may then also generate an instruction for the patient (310).

In some examples, processor 170 may provide the lung function test as a guide of progress for the patient. Processor 170 may measure the lung function and simply generate an alert for presentation to the patient if the lung function test indicates abnormal, or improper, lung function. The test may be initialized by the user via selection of a user input of computing device 22 or initialized automatically by computing device 22 or PMDD 14. In this manner, the lung function test may be manual or automatic as feedback for disease progress.

In other examples, improper lung function may trigger additional processes to manage patient health. For example, if an improper lung function is determined, computing device 22 may then being to track environmental conditions. Since diminished lung function may put the patient at increased risk to environmental conditions, computing device 22 may reduce patient exposure to environmental problems may tracking these conditions and alerting the patient of any potential issues. Improper lung function may also cause computing device 22 to instruct the patient to take a dose of medication.

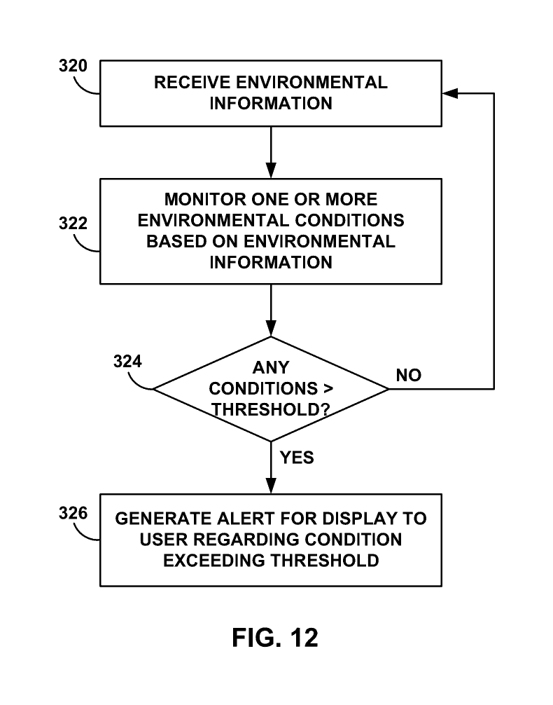

FIG. 12 is a flow diagram of an example process for monitoring one or more environmental conditions relevant to a patient. The example processor of FIG. 12 is described with respect to components (e.g., processor 170, UI device 174, and communication unit 176) of computing device 22 of FIGS. 1 and 7 and PMDD 14 (although any other PMDD may be used). In some examples, applications 186 may perform these features via processor 170. However, in other examples, one or more aspects of the processor of FIG. 12 may be performed by a different computing device or a PMDD in other examples.

As shown in FIG. 12, processor 170 receives environmental information from a remote computing device (320). The environmental information may be received from a governmental agency server, a weather service server, a company server, a clinician server, or any other networked computing device configured to notify users of changes to an environmental situation. The specific information received from such servers or services may be based on the geo-location of computing device 22 that is reported to the services or otherwise used to receive appropriate location information. Environmental information may be transmitted to computing device 22 in response to receiving geo-location information from computing device 22. In other examples, computing device 22 may receive generic environmental information and determine when computing device 22 is located in any alert areas identified by the environmental information. Processor 170 may then monitor one or more environmental conditions based on the environmental information (322) and determine if any of the environmental conditions (e.g., one or more condition parameter values) exceed their respective thresholds (324). If none of the environmental conditions exceeds the respective threshold (“NO” branch of block 324), processor 170 may continue to receive environmental information (320). If one or more of the environmental conditions exceeds the respective threshold (“YES” branch of block 324), processor 170 may generate an alert for display to a user regarding the environmental condition or conditions exceeding the respective threshold (326). The user may be the patient associated with computing device 22 and PMDD 14 in one example. In addition or alternatively, the user may be the clinician monitoring the patient associated with PMDD 14 in another example. The process of FIG. 12 is described with respect to environmental conditions, but the process similarly applies to patient specific conditions or a combination of different types of disease-relevant conditions (e.g., environmental conditions and patient-specific conditions). For example, the process of FIG. 12 may be used to determine if any condition parameter values for respective patient conditions exceed respective condition thresholds and generate an alert regarding any patient condition that exceeded the respective condition threshold.

In other examples, processor 170 may only generate the alert for the user when a threshold number of disease-relevant conditions (e.g., environmental conditions and/or patient specific conditions) exceed the respective thresholds. In other words, the alert may only be generated if a certain number of disease-relevant conditions (e.g., environmental conditions and/or patient specific conditions) indicate that the environment may cause a problem for the patient or patient activities may contribute to a disease state. In some examples, each of the disease-relevant conditions may be weighted and/or weighted based on how many of the respective thresholds are exceeded. For example, a total score may be calculated based on the weighted score of each environmental condition to account for some environmental conditions that may be more problematic for the patient than other environmental conditions. Such weighting may be applied to any disease-relevant conditions, such as patient specific conditions. Processor 170 may, in some examples, monitor the time of each disease-relevant condition and only generate the alert of the disease-relevant condition was measured within a predetermined period of time of the current time. This determination may prevent out of date information from being used in alerting the patient. In addition to alerting the patient, disease-relevant conditions exceeding thresholds may also be transmitted to a clinician form patient monitoring.

Many different disease-relevant conditions may be tracked any used to help the patient manage their respiratory disorder. Example environmental conditions that computing device 22 may track may include pollen counts (e.g., high pollen counts), one or more allergens, pollen counts, one or more air irritants, the presence of smog, air pollution status, an air quality index, high altitude of the patient, air temperature, heat index, wind-chill, humidity, construction areas, or any other item that may affect respiratory disorder of the patient. In some examples, a patient specific condition may include conditions related to physical activity, a breathing rate, a heart rate, a perspiration rate, a caloric burn rate, a pulmonary function, a sleep state of the patient, or other conditions sensed by one or more devices associated with the patient. Physical activity or exertion of the patient may be detected via an accelerometer or other activity device in communication with computing device 22.

Environmental condition inputs may be from sensors on computing device 22 (e.g., geo-location sensors, temperature sensors, and/or altitude sensors), wearable sensors (e.g., wearable sensors worn by the patient) in communication with computing device 22, weather applications executing on computing device 22, push notifications from one or more networked services, a governmental agency, a clinician's office, or any other source of information. The specific information received from such devices or services may be based on the geo-location of computing device 22 that is detected by computing device 22 (e.g., via a GPS device) and reported to the services or otherwise used to receive appropriate location information. Patient specific conditions may similarly be detected via external sensors, wearable sensors, and/or sensors contained within computing device 22. Computing device 22 may be configured to receive or acquire information related to the disease-relevant conditions in response to determining that an actuation of medication occurred. Computing device 22 may also receive or acquire information related to the disease-relevant conditions periodically, continually, or otherwise independent from when an actuation occurred. The thresholds used for each disease-relevant condition may be determined or set by a clinician or other healthcare professional. For example, a clinician may transmit a table or database that includes each of the respective thresholds, any weighting factors for the thresholds, the number of thresholds that need to be exceed to trigger an alert to the patient, or any other relevant information. In some examples, the patient may have some degree of control over the threshold to fine tune feedback and therapy.

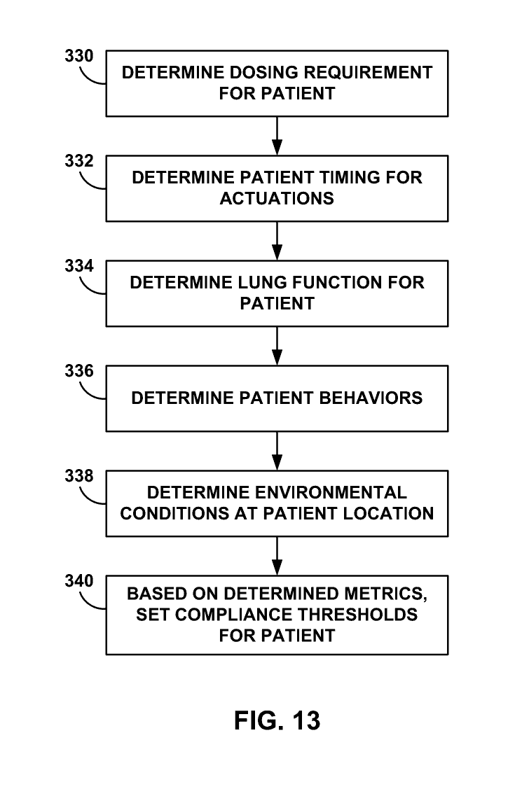

FIG. 13 is a flow diagram of an example process for determining compliance thresholds for a patient. The example processor of FIG. 13 is described with respect to components (e.g., processor 170, UI device 174, and communication unit 176) of computing device 22 of FIGS. 1 and 7 and PMDD 14 (although any other PMDD may be used). In some examples, applications 186 may perform these features via processor 170. However, in other examples, one or more aspects of the processor of FIG. 9 may be performed by a different computing device or a PMDD in other examples.

As shown in FIG. 13, processor 170 determines several metrics regarding patient health, drug delivery, and environmental conditions to establish overall compliance thresholds for determining when the patient is complying with established therapy protocols. Processor 170 may determine a dosing requirement for the patient (330) and determine the normal patient timing for medication actuations using PMDD 14 (332). Processor 170 may also determine current lung function for the patient to track any changes over time (334) and determine typical patient behaviors or activities to establish a baseline (336). Processor 170 may also determine the typical environmental conditions at the patient location or establish environmental condition thresholds based on patient health (338). Based on the determined metrics for the patient, processor 170 may set the compliance thresholds used for each metric to determine when the metric exceeds the respective thresholds that are patient-specific (340).

In the processes of any of FIGS. 11-15, thresholds may be set by a clinician or patient. For example, the clinician may set a threshold for when air quality triggers an alert to the patient or when physical activity triggers an alert to the patient. Computing device 22 may provide a settings section where the thresholds can be viewed along with current data points in relation to the thresholds. In some examples, one or more of the thresholds for the processes of FIGS. 11-15 may be dynamic based on the current patient condition. For example, if recent actuations of the PMDD have been improper, computing device 22 may decrease the threshold for pollen count because the patient may be more susceptible to pollen given the lack of medication recently received. In this manner, computing device 22 may execute one or more algorithms to adjust thresholds in an dynamic manner to mirror the condition of the patient.

FIG. 14 is a conceptual diagram of an example user interface 350 providing data related to patient initiated actuations. As shown in FIG. 14, computing device 22 or any other computing device disclosed herein may generate user interface 350 to provide flow data to the patient or other user. User interface 350 may include a connection status 352 to PMDD 14, for example. Graph 354 provides flow data 356 over time as measured within PMDD 14 and actuation data 358 as a representation of the actuation of medication. Flow data 356 and actuation data 358 are matched in time and overlaid to show the timing of the actuation with respect to the air flow during an inhalation. Actuation data 358 showing the actuation during the peak flow of the inhalation in flow data 356 may indicate proper or “good” actuation timing from the patient.

In some examples, proper actuation timing (e.g., good timing) may be determined when the slope of flow data 356 is positive (e.g., increasing flow rate) at the time the actuation data 358 indicates the actuation was initiated. In addition, or alternatively, improper actuation may be determined by computing device 22 when the slope of flow data 356 is negative at the time actuation data 358 indicates the actuation was initiated. The timing of the actuation represented by actuation data 358 with respect to flow data 356 may thus be used to determine when an actuation was appropriately timed. In other examples, proper actuation timing may be determined by an actuation overlapping with the peak inhalation flow rate (as shown in FIG. 14), an actuation occurring entirely within the positive slope of flow data 356 (e.g., during increasing inhalation flow rates), or actuation being initiated within a predetermined threshold time from the beginning of the inhalation.

Although no flow units are provided in the example of user interface 350, flow rate or flow velocity may be quantified in other examples. Timestamp 360 may indicate the time and date of the actuation and/or inhalation. User interface 350 may also indicate whether the actuation was “good” or “bad”. Computing device 22 may allow the patient to scroll between successive actuations and inhalations to review past actuations.

FIG. 15 is a conceptual diagram of an example user interface 370 providing qualified actuation information of actuations over time. As shown in FIG. 15, computing device 22 or any other computing device disclosed herein may generate user interface 350 to provide flow data to the patient or other user. User interface 370 may provide a review of actuations over time and qualify each actuation as proper (e.g., “good”) or improper (e.g., “bad”). Ratio status 372 indicates the percentage of actuations that have been good and the percentage of actuations that have been bad over the given time period.

Graph 374 plots each actuation at its given date and time. Proper actuations 378 are visually indicated by shape and/or color (e.g., green for “good” actuations). Conversely, improper actuations 376 are visually indicated by shape and/or color (e.g., red for “bad” actuations). The visual indication of proper and improper actuations quickly provides feedback to the patient regarding actuation technique and possible lack of medication on days during improper actuations. In other examples, each plotted actuation may be displayed for a day without the time of day. Computing device 22 may provide inputs that allow the user to sort actuations based on proper and improper actuations, time of day, or any other quality.

In some examples, each actuation 378 and 376 within graph 374 may be selected by the user to view additional information regarding the selected actuation. For example, additional details related to the time of the actuation, flow data from the actuation, and/or the type and dose of medication delivered may be provided. In addition, computing device 22 may allow the patient to enter a note regarding the particular actuation and/or view previously entered notes. Notes may allow the patient to track any unusual circumstances or explain why multiple actuations were performed in a short period of time, for example. In addition, computing device 22 may store other recorded information at the time of the actuation, such as patient activity, heart rate, geo-location of computing device 22, one or more environmental conditions, or any other related information disclosed herein.

Utilizing user interfaces of collected data such as user interface 370, computing device 22 may provide data and trends of drug usage and/or pulmonary function over time (e.g., by hour, day, week, month, year, or lifetime). In other words, computing device 22 may provide trend data in numerical and/or graph form of drug use and pulmonary function.

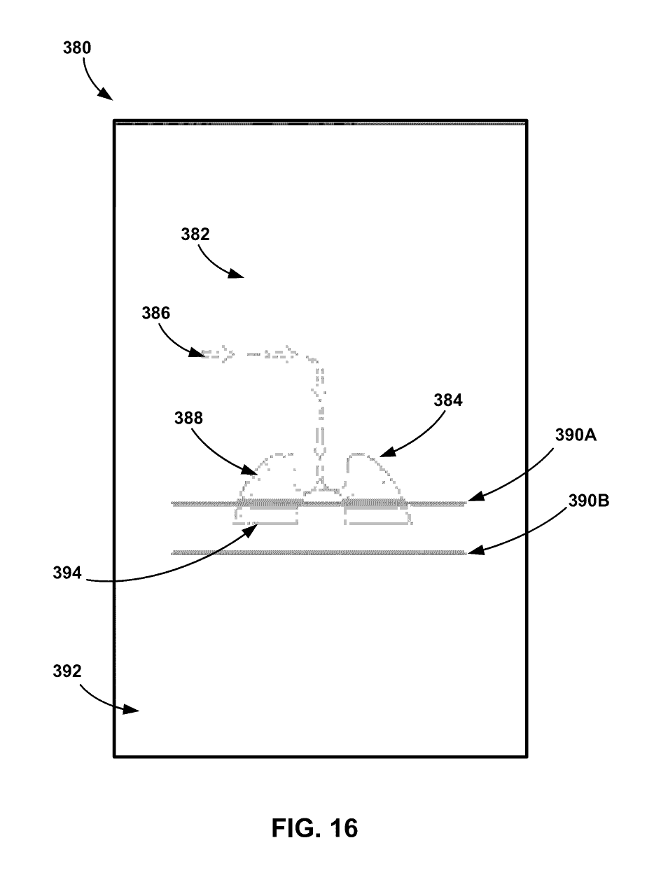

FIG. 16 is a conceptual diagram of an example user interface 380 providing an indication of effective actuation timing for a detected inhalation of a patient. As shown in FIG. 16, computing device 22 or any other computing device disclosed herein may generate, for display, user interface 380 to provide an indication of inhalation progress and suggested timing for which the user should actuation the medication from the PMDD. In this manner, user interface 380 may assist the user in training the user to actuate medication deliver at the appropriate time during inhalation.

User interface 380 may include a user icon 382 with a representation of the airway and lungs 384 over the user icon 382. Minimum volume threshold 390A may indicate the minimum estimated lung volume for effective medication delivery and maximum volume threshold 390B may indicate the maximum estimated lung volume for effective medication delivery. The area between minimum volume threshold 390A and maximum volume threshold 390B may define the target zone (e.g., the “green zone” as shown in FIG. 16) for the user to actuate medication delivery from the PMDD. Instructions 392 may provide instructions for the user on when to actuate the medication canister according to the information shown in user interface 380, e.g. “Actuate in Green Zone.”

The flow sensor associated with the PMDD may detect airflow of an inhalation and the PMDD may transmit the airflow data to computing device 22, for example. Based on the received airflow data, computing device 22 may generate a representation of the inhalation in user interface 380. For example, the representation of airflow may be shown as arrows 386 indicating the direction of airflow (e.g., arrows pointing to lungs 384 to indicate an inhalation, and computing device 22 may shade or fill the area of lungs 384 according to the detected airflow from the flow sensor. Lung volume 388 is the representation of the progress of the inhalation, and volume level 394 shows the boundary of the lung volume 388 as the inhalation progresses. In other words, volume level 394 may move to show increasing lung volume during the inhalation. Volume level 294 shown as between minimum volume threshold 390A and maximum volume threshold 390B indicates the appropriate, or proper, time in which the user should actuate the medication canister. Minimum volume threshold 390A and maximum volume threshold 390B may be set based on previous inhalations from the patient, patient input, and/or clinician input.

In some examples, computing device 22 may receive an indication of when the actuation is detected (e.g., from an actuation sensor associated with the PMDD) and update user interface 380 to present an indication that the actuation occurred within the target zone for actuation (e.g., a proper or effective actuation) or outside of the target zone for actuation (e.g., an improper or ineffective actuation). The shaded area of lung volume 388 and volume level 394 may correspond to calculated, or estimated, volumes of inhalation based on the airflow detected at the PMDD. However, in other examples, the shaded area of lung volume 388 and volume level 394 may not necessarily correspond to the volume of air of the inhalation. Instead, lung volume 388 and volume level 394 may represent a timing based on initial detected inhalation air flow, appropriate actuation based on changes in measured flow velocity (e.g., actuation may be effective when it occurs during peak air velocities or air flow rates), or any other metrics that may represent a progress or status of an inhalation from the patient. In this manner, lung volume 388 and volume level 394 may, or may not, actually represent air volume when representing the progress of the inhalation for the patient.

The following examples illustrate example methods, devices, and systems described herein. Example 1 a method comprising sensing, by a sensor, air flow within a portion of a pulmonary medication dosing device and transmitting, by a processor, a signal indicative of the sensed air flow to a computing device, wherein information associated with use of the pulmonary medication dosing device is generated by the computing device and based on the transmitted signal.

Example 2, the method of example 1, further comprising detecting, by an actuation sensor, an actuation of the medication canister, generating a signal indicative of the detected actuation of the medication canister, and transmitting, by the processor, the signal indicative of the detected actuation.

Example 3, the method of example 2, wherein the signal indicative of the sensed air flow indicates a first timing of a plurality of airflow values and the signal indicative of the detected actuation indicates a second timing of the actuation of the medication canister, and wherein correlation of the first timing of the plurality of airflow values and the second timing of the actuation of the medication canister indicates an effectiveness of the detected actuation in delivering medication from the medication canister to a patient via the sensed air flow.

Example 4, the method of example 3, further comprising correlating the first timing of the plurality of airflow values to the second timing of the actuation of the medication canister, and determining, based on the correlation, the effectiveness of the detected actuation in delivering medication from the medication canister to a patient via the sensed air flow.

Example 5, the method of example 4, further comprising presenting an indication of the effectiveness of the detected actuation in delivering medication from the medication canister to a patient via the sensed air flow.

Example 6, the method of any of examples 2 through 5, wherein transmitting the signal indicative of the sensed air flow comprises transmitting the signal indicative of the sensed air flow to a mobile computing device in communication with the pulmonary medication dosing device, and transmitting the signal indicative of the detected actuation of the medication canister comprises transmitting the signal indicative of the detected actuation of the medication canister to the mobile computing device in communication with the pulmonary medication dosing device.

Example 7, the method of any of examples 2 through 6, further comprising generating a drug delivery log based the signal indicative of the detected actuation of the medication canister, wherein the drug delivery log comprises an indication of one or more detected actuations of the medication canister and a time at which each of the respective one or more detected actuations occurred, and outputting, for display, information indicative of the drug delivery log.

Example 8, the method of any of examples 1 through 7, wherein an attachable housing is configured to be attached to a housing of the pulmonary medication dosing device, and wherein the attachable housing comprises the sensor and the processor.

Example 9, the method of any of examples 1 through 8, wherein the pulmonary medication dosing device comprises the sensor and the processor.

Example 10, the method of any of examples 1 through 9, wherein the computing device is a mobile computing device.

Example 11, the method of any of examples 1 through 10, further comprising calculating one or more parameters of pulmonary function from the signal indicative of the sensed air flow, and outputting, for display, the one or more parameters.

Example 12, the method of any of examples 1 through 11, further comprising receiving one or more condition thresholds from a clinician, wherein each of the one or more condition thresholds are associated with a respective environmental condition or patient condition of a patient associated with the pulmonary medication dosing device, storing, in a memory, the one or more condition thresholds, receiving one or more condition parameter values for the respective environmental condition or patient condition, determining that at least one of the one or more condition parameter values exceed a respective condition threshold, and responsive to the determination, generate, for display, an alert that instructs a patient to take a dose of medication from the pulmonary medication dosing device.

Example 13, the method of any of examples 1 through 12, wherein an alert that instructs a user to actuate a medication canister associated with the pulmonary medication dosing device is generated based on the transmitted signal.

Example 14, the method of any of examples 1 through 13, further comprising generating, by the processor, an alert that instructs the user to actuate the medication canister.

Example 15, the method of any of examples 1 through 14, further comprising delivering an alert that instructs the user to actuate the medication canister, wherein the alert comprises at least one of a visual cue, an audible cue, and a tactile cue.

Example 16, the method of any of examples 1 through 15, further comprising generating, by the processor, the signal indicative of the sensed air flow.

Example 17, a computer-readable storage medium comprising instructions that, when executed by one or more processors, cause the one or more processors to perform the method of any of examples 1-16.

Example 18, a system configured to perform the method of any of claims 1-16.

Example 19, a system comprising means to perform the method of any of claims 1-16.

Example 20, a system comprising a housing configured to accept a medication canister containing a medication, the housing comprising a dispensing portion, a sensor configured to sense air flow within the dispensing portion, and a processor configured to transmit a signal indicative of the sensed air flow to a computing device, wherein information associated with use of the pulmonary medication dosing device is generated by the computing device and based on the transmitted signal.

Example 21, the system of example 20, wherein an alert that instructs a user to actuate a medication canister associated with the housing is generated based on the transmitted signal.

Example 22, the system of any of examples 20 and 21, wherein the processor is configured to generate the signal indicative of the sensed air flow.

Example 23, the system of any of examples 20 through 22, wherein the sensor is a first sensor, and wherein the system further comprises a second sensor configured to detect actuation of the medication canister, and wherein the processor is configured to generate a signal indicative of the detected actuation of the medication canister and transmit the signal indicative of the detected actuation.

Example 24, the system of example 11, wherein the processor is configured to transmit the signal indicative of the sensed air flow to a mobile computing device in communication with the processor, and transmit the signal indicative of the detected actuation of the medication canister to the mobile computing device in communication with the processor.

Example 25, the system of any of examples 20 through 24, wherein the processor is configured to generate an alert that instructs the user to actuate the medication canister.

Example 26, the system of any of examples 20 through 25, further comprising one or more output devices configured to deliver an alert that instructs the user to actuate the medication canister, wherein the alert comprises at least one of a visual cue, an audible cue, and a tactile cue.

Example 27, a method comprising receiving, by a processor of a computing device, a signal indicative of sensed air flow within a portion of a pulmonary medication dosing device in communication with the processor, and generating, by the processor and based on the signal, information associated with use of the pulmonary medication dosing device.

Example 28, the method of example 27, wherein generating the information comprises generating an alert that instructs a user to actuate a medication canister associated with the pulmonary medical dosing device.

Example 29, the method of example 28, further comprising delivering the alert to the user.

Example 30, the method of any of examples 28 and 29, wherein the alert comprises at least one of a visual cue, an audible cue, and a tactile cue.

Example 31, the method of any of examples, 27 through 30, further comprising receiving a signal indicative of a detected actuation of the medication canister and storing, in a memory of the computing device, a representation of the signal indicative of the detected actuation of the medication canister.

Example 32, the method of any of examples 27 through 31, further comprising generating a drug delivery log based on one or more received signals indicative of respective detected actuation of the medication canister and transmitting, from the computing device, the drug delivery log.

Example 33, a computing device comprising one or more processors configured to receive, from a pulmonary medication dosing device, a signal indicative of sensed air flow within a portion of the pulmonary medication dosing device, and generate, based on the signal, information associated with use of the pulmonary medication dosing device.

Example 34, the computing device of example 33, wherein the one or more processors are configured to generate an alert that instructs a user to actuate a medication canister associated with the pulmonary medication dosing device.

Example 35, the computing device of example 34, further comprising one or more output devices configured to deliver the alert to the user.

Example 36, the computing device of any of examples 34 and 35, wherein the alert comprises at least one of a visual cue, an audible cue, and a tactile cue.

Example 37, the computing device of any of examples 33 through 36, wherein the one or more processors are configured to receiving a signal indicative of a detected actuation of the medication canister, and wherein the computing device comprises a memory configured to store a representation of the signal indicative of the detected actuation of the medication canister.

Example 38, the computing device of any of examples 33 through 37, wherein the one or more processors are configured to generate a drug delivery log based on one or more received signals indicative of respective detected actuation of the medication canister and transmit, from the computing device, the drug delivery log.

Example 39, a computer-readable storage medium comprising instructions that, when executed by one or more processors of a computing device, cause the one or more processors to perform the method of any of examples 33 through 37.

Example 40, a system comprising means to perform the method of any of examples 33 through 37.

Example 41, a method comprising sensing air flow within a portion of a pulmonary medication dosing device, and transmitting, by a processor, a signal indicative of the sensed air flow, wherein one or more parameters of pulmonary function can be calculated, for display, from the signal.

Example 42, the method of example 41, wherein the processor is a first processor, and wherein the method further comprises calculating, by a second processor receiving the signal, the one or more parameters of pulmonary function from the signal, and outputting, for display, the one or more parameters.

Example 43, the method of example 42, further comprising displaying, by a display device, the one or more parameters.

Example 44, the method of any of examples 41 through 43, wherein transmitting the signal comprises transmitting, by the processor and via a network, the signal to a networked server.

Example 45, the method of example 44, wherein the networked server is a cloud-based server system.

Example 46, a method comprising sensing air flow within a portion of a pulmonary medication dosing device, transmitting, by a first processor housed in the pulmonary medication dosing device, a signal indicative of the sensed air flow to a computing device, receiving, by a second processor of the computing device, the signal indicative of sensed air flow within the portion of the pulmonary medication dosing device, and generating, by the second processor and based on the signal, information associated with use of the pulmonary medication dosing device.

Example 47, the method of example 46, further comprising generating, by the second processor, a graphical representation of the information representative of the sensed air flow, and presenting, by an output device of the computing device, the graphical representation.

Example 48, the method of any of examples 46 and 47, further comprising transmitting, by the second processor of the computing device, the generated information to a remote server associated with a clinician monitoring the status of a patient utilizing the pulmonary medication dosing device.

Example 49, a device comprising an attachable housing configured to be attached to a metered dose inhaler housing, the metered dose inhaler housing configured to receive a medication canister, a processor housed by the attachable housing, an air flow sensor coupled to the attachable housing and positionable within an air flow channel of the metered dose inhaler housing, wherein the air flow sensor is configured to detect air flow related to a patient breath and generate air flow data representative of the detected air flow, and a communication unit housed by the attachable housing, wherein the processor is configured to receive the air flow data from the air flow sensor and transmit, via the communication unit, at least a portion of the air flow data to a computing device.

Example 50, the device of example 49, wherein the communication unit is configured to transmit the air flow data via a Bluetooth communication protocol.

Example 51, the device of any of examples 49 and 50, wherein the communication unit is configured to transmit, via a cellular communication protocol, the air flow data over a cellular communication network to the computing device.

Example 52, a method comprising receiving, by a processor of a computing device, an air flow signal indicative of sensed air flow within a portion of a pulmonary medication dosing device in communication with the processor, receiving, by the processor, an actuation signal indicative of an actuation of a medication canister associated with the pulmonary medication dosing device, determining, based on the timing of the actuation with respect to the sensed air flow, an effectiveness of the actuation in delivering medication from the medication canister to a patient via the sensed air flow, and generating, by the processor and based on the determination, an indication of the effectiveness of the actuation.

Example 53, the method of example 52, further comprising receiving environmental information representative of one or more environmental conditions to which a patient associated with the pulmonary medical dosing device is exposed, storing the environmental information, and generating, for display, a representation of the environmental conditions.

Example 54, the method of example 53, further comprising correlating the one or more environmental conditions to the actuation and generating, for display, a representation of the correlation of the one or more environmental conditions to the actuation.

Example 55, the method of any of examples 53 and 54, wherein the one or more environmental conditions comprise one or more of a global position system location, an air quality index, an air temperature, a smog index, a pollen index, an altitude, a time of day, a construction level index, or an air humidity.

Example 56, the method of any of examples 53 through 55, wherein the one or more environmental conditions are received from a networked server that generated an indication of the one or more environmental conditions.

Example 57, the method of any of examples 53 through 56, wherein the one or more environmental conditions are received from a sensor housed by a wearable computing device associated with a patient utilizing the pulmonary medication dosing device.

Example 58, the method of any of examples 52 through 57, further comprising receiving patient information representative of one or more patient specific conditions, storing the patient information, and generating, for display, a representation of the patient conditions.

Example 59, the method of example 58, further comprising correlating the one or more patient conditions to the actuation and generating, for display, a representation of the correlation of the one or more patient conditions to the actuation.

Example 60, the method of any of examples 58 and 59, wherein the one or more patient conditions comprise at least one of a physical activity, a breathing rate, a heart rate, a perspiration rate, a caloric burn rate, a pulmonary function, or a sleep state.

Example 61, the method of any of examples 58 through 60, wherein the one or more patient conditions are received from a sensor housed by a wearable computing device associated with a patient utilizing the pulmonary medication dosing device.

Example 62, the method of any of examples 52 through 61, further comprising receiving one or more condition thresholds from a clinician, wherein each of the one or more condition thresholds are associated with a respective environmental condition or patient condition of a patient associated with the pulmonary medication dosing device, storing, in a memory of the computing device, the one or more condition thresholds, receiving condition parameters for the respective environmental condition or patient condition, determining that at least one of the condition parameters exceed a respective condition threshold, and responsive to the determination, generate, for display, an alert that instructs a patient to take a dose of medication from the pulmonary medication dosing device.

The disclosure also contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to any of PMDD 14, 50, 80, and 140, computing device 22, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. For example, any of the techniques or processes described herein may be performed within one device or at least partially distributed amongst two or more devices, such as between PMDD 14 and computing device 22, for example. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

1-16. (canceled)

17. A system comprising:
a sensor configured to sense air flow within a portion of a pulmonary medication dosing device;
one or more processors configured to transmit, to a computing device, a signal indicative of the sensed air flow, wherein information associated with use of the pulmonary medication dosing device is generated by the computing device and based on the transmitted signal; and
an attachable housing configured to be attached to a housing of the pulmonary medication dosing device, wherein the attachable housing comprises the sensor and at least one of the one or more processors, and wherein the attachable housing is configured to dispose the sensor within a channel defined within the housing of the pulmonary medication dosing device and between the a wall of the housing of the pulmonary medication dosing device and a medication canister when the attachable housing is attached to the housing of the pulmonary medication dosing device.

18. The system of claim 17, wherein a portion of the housing of the pulmonary medical dosing device is configured to be positioned between a portion of the attachable housing and the medication canister when the sensor is within the channel.

19. The system of claim 17, wherein the attachable housing comprises an actuation sensor configured to detect an actuation of the medication canister.

20. The system of claim 19, wherein the actuation sensor comprises an optical sensor configured to detect movement of the medication canister.

21. The system of claim 17, wherein the attachable housing comprises a visual indicator configured to illuminate in response to an actuation of the medication canister timed correctly with a detected inhalation.

22. The system of claim 17, wherein the attachable housing is configured to snap onto an outside of the housing of the pulmonary medication dosing device.

23. The system of claim 17, wherein the attachable housing comprises one or more clips configured to clip the attachable housing onto a portion of the housing of the pulmonary medication dosing device.

24. The system of claim 17, wherein the attachable housing does not completely surround the medication canister.

25. The system of claim 17, wherein the housing of the pulmonary medication dosing device is a first housing of a first pulmonary medical dosing device, and wherein the attachable housing is transferable to attach to a second housing of a second pulmonary medication dosing device.

26. The system of claim 17, further comprising a mobile computing device as the computing device, the mobile computing device comprising a memory, wherein the mobile computing device is configured to:
receive one or more condition thresholds from a clinician, wherein each of the one or more condition thresholds are associated with a respective environmental condition or patient condition of a patient associated with the pulmonary medication dosing device;
store, in the memory, the one or more condition thresholds;
receive one or more condition parameter values for the respective environmental condition or patient condition;
determine that at least one of the one or more condition parameter values exceed a respective condition threshold; and
responsive to the determination, generate, for display, an alert that instructs a patient to take a dose of medication from the pulmonary medication dosing device.

27. The system of claim 26, wherein the attachable housing comprises an output device configured to display the alert.

28. The system of claim 17, wherein the one or more processors are configured to:
generate a drug delivery log based a signal indicative of a detected actuation of the medication canister, wherein the drug delivery log comprises an indication of one or more detected actuations of the medication canister and a time at which each of the respective one or more detected actuations occurred; and
output, for display, information indicative of the drug delivery log.

29. The system of claim 17, wherein the one or more processors are configured to:
receive environmental information representative of one or more environmental conditions to which a patient associated with the pulmonary medical dosing device is exposed;
correlate the one or more environmental conditions to a detected actuation of the medication canister; and
generate, for display, a representation of the correlation of the one or more environmental conditions to the detected actuation, wherein the one or more environmental conditions comprise one or more of a global position system location, an air quality index, an air temperature, a smog index, a pollen index, an altitude, a time of day, a construction level index, or an air humidity.

30. The system of claim 17, further comprising the computing device, wherein the computing device comprises a display device and the computing device is configured to:
control the display device to display a representation of lungs of a patient, a minimum volume threshold that indicates a minimum lung volume for effective medication delivery, a maximum volume threshold that indicates a maximum lung volume for effective medication delivery, and a representation of a lung volume level with respect to the representation of the lungs;
determine, based on the signal indicative of the sensed air flow, changes to the lung volume of the patient; and
control the display device to change the representation of the lung volume based on the changes to the lung volume of the patient, wherein proper actuation of the medication canister occurs when the representation of the lung volume is between the minimum volume threshold and the maximum volume threshold.

31. The system of claim 17, wherein:
the signal indicative of the sensed air flow indicates a first timing of a plurality of airflow values and a signal indicative of a detected actuation indicates a second timing of the actuation of the medication canister;
correlation of the first timing of the plurality of airflow values and the second timing of the actuation of the medication canister indicates an effectiveness of the detected actuation in delivering medication from the medication canister to a patient via the sensed air flow; and
the one or more processors are configured to:
correlate the first timing of the plurality of airflow values to the second timing of the actuation of the medication canister;
determine, based on the correlation, the effectiveness of the detected actuation in delivering medication from the medication canister to a patient via the sensed air flow; and
output, for presentation to a user, an indication of the effectiveness of the detected actuation in delivering medication from the medication canister to a patient via the sensed air flow.

32. The system of claim 31, wherein the one or more processors are configured to:
qualify, for each of a plurality of detected actuations, effectiveness of a detected actuation as one of proper or improper; and
output, for presentation to the user, visual indications for each of the plurality of detected actuations that visually distinguish each respective detected actuation as one of proper or improper.

33. The system of claim 32, wherein the one or more processors are configured to:
generate, for the plurality of detected actuations, a ratio of proper actuations to improper actuations for a period of time; and
output, for display to the user, the ratio of proper actuations to improper actuations for a period of time.

34. The system of claim 17, wherein the one or more processors are configured to:
receive actuation information representative of one or more actuations of the medication canister;
determine, based on the actuation information, a number of actuations of the medication canister that occurred within a period of time;
compare the number of actuations that occurred within the period of time to a threshold; and
generate, for display to a user, an alert indicating that the number of actuations within the period of time exceeds the threshold.

35. A method comprising:
sensing, by a sensor of an attachable housing, air flow within a portion of a pulmonary medication dosing device, wherein the attachable housing is configured to be attached to a housing of the pulmonary medication dosing device; and
transmitting, by one or more processors of the attachable housing, a signal indicative of the sensed air flow to a computing device, wherein:
information associated with use of the pulmonary medication dosing device is generated by the computing device and based on the transmitted signal, and
the attachable housing is configured to dispose the sensor within a channel defined within the housing of the pulmonary medication dosing device and between the a wall of the housing of the pulmonary medication dosing device and a medication canister when the attachable housing is attached to the housing of the pulmonary medication dosing device.

36. The method of claim 35, further comprising detecting, by an actuation sensor of the attachable housing, an actuation of the medication canister.

37. The method of claim 35, further comprising controlling a visual indicator of the attachable housing to illuminate in response to an actuation of the medication canister timed correctly with a detected inhalation.

38. The method of claim 35, wherein the computing device comprises a memory, and wherein the method further comprises:
receiving, by the computing device, one or more condition thresholds from a clinician, wherein each of the one or more condition thresholds are associated with a respective environmental condition or patient condition of a patient associated with the pulmonary medication dosing device;
storing, in the memory, the one or more condition thresholds;
receiving, by the computing device, one or more condition parameter values for the respective environmental condition or patient condition;
determining, by the computing device, that at least one of the one or more condition parameter values exceed a respective condition threshold; and
responsive to the determination, generating, by the computing device and for display, an alert that instructs a patient to take a dose of medication from the pulmonary medication dosing device.

39. The method of claim 35, further comprising:
generating a drug delivery log based a signal indicative of a detected actuation of the medication canister, wherein the drug delivery log comprises an indication of one or more detected actuations of the medication canister and a time at which each of the respective one or more detected actuations occurred; and
outputting, for display, information indicative of the drug delivery log.

40. The method of claim 35, further comprising:
receiving environmental information representative of one or more environmental conditions to which a patient associated with the pulmonary medical dosing device is exposed;
correlating the one or more environmental conditions to a detected actuation of the medication canister; and
generating, for display, a representation of the correlation of the one or more environmental conditions to the detected actuation, wherein the one or more environmental conditions comprise one or more of a global position system location, an air quality index, an air temperature, a smog index, a pollen index, an altitude, a time of day, a construction level index, or an air humidity.

41. The method of claim 35, wherein the computing device comprises a display device, and wherein the method further comprises:
controlling the display device to display a representation of lungs of a patient, a minimum volume threshold that indicates a minimum lung volume for effective medication delivery, a maximum volume threshold that indicates a maximum lung volume for effective medication delivery, and a representation of a lung volume level with respect to the representation of the lungs;
determining, based on the signal indicative of the sensed air flow, changes to the lung volume of the patient; and
controlling the display device to change the representation of the lung volume based on the changes to the lung volume of the patient, wherein proper actuation of the medication canister occurs when the representation of the lung volume is between the minimum volume threshold and the maximum volume threshold.

42. The method of claim 35, wherein:
the signal indicative of the sensed air flow indicates a first timing of a plurality of airflow values and a signal indicative of a detected actuation indicates a second timing of the actuation of the medication canister;
correlation of the first timing of the plurality of airflow values and the second timing of the actuation of the medication canister indicates an effectiveness of the detected actuation in delivering medication from the medication canister to a patient via the sensed air flow; and
the method further comprises:
correlating the first timing of the plurality of airflow values to the second timing of the actuation of the medication canister;
determining, based on the correlation, the effectiveness of the detected actuation in delivering medication from the medication canister to a patient via the sensed air flow; and
outputting, for presentation to a user, an indication of the effectiveness of the detected actuation in delivering medication from the medication canister to a patient via the sensed air flow.

43. The method of claim 42, further comprising:
qualifying, for each of a plurality of detected actuations, effectiveness of a detected actuation as one of proper or improper; and
outputting, for presentation to the user, visual indications for each of the plurality of detected actuations that visually distinguish each respective detected actuation as one of proper or improper.

44. The method of claim 35, further comprising:
receiving actuation information representative of one or more actuations of the medication canister;
determining, based on the actuation information, a number of actuations of the medication canister that occurred within a period of time;
comparing the number of actuations that occurred within the period of time to a threshold; and
generating, for display to a user, an alert indicating that the number of actuations within the period of time exceeds the threshold.

45. A system comprising:
means for sensing air flow within a portion of a pulmonary medication dosing device;
means for transmitting, to a computing device, a signal indicative of the sensed air flow, wherein information associated with use of the pulmonary medication dosing device is generated by the computing device and based on the transmitted signal; and
an attachable housing comprising the means for sensing air flow and the means for transmitting the signal, the attachable housing comprising a means for attaching the attachable housing to a housing of the pulmonary medication dosing device, wherein the attachable housing is configured to dispose the means for sensing within a channel defined within the housing of the pulmonary medication dosing device and between the a wall of the housing of the pulmonary medication dosing device and a medication canister when the attachable housing is attached to the housing of the pulmonary medication dosing device.
